(19) United States
(12) Patent Application Publication
(10) Pub. No.: US 2025/0241904 A1
(43) Pub. Date: Jul. 31, 2025

(54) COMPOSITION AND METHOD FOR PREVENTION AND TREATMENT OF CUTANEOUS RADIATION INJURY

(71) Applicant: Rythera Therapeutics, Inc., Deerfield Beach, FL (US)

(72) Inventor: Reginald Hardy, Ft. Lauderdale, FL (US)

(73) Assignee: Rythera Therapeutics, Inc., Ft. Lauderdale, FL (US)

(21) Appl. No.: 19/087,474

(22) Filed: Mar. 22, 2025

Related U.S. Application Data
(63) Continuation of application No. PCT/US2023/074914, filed on Sep. 22, 2023.
(60) Provisional application No. 63/408,997, filed on Sep. 22, 2022.

(51) Int. Cl. A61K 31/4545; A61K 9/00; A61K 9/06; A61K 9/08; A61K 31/135; A61P 17/02

(52) U.S. Cl. CPC A61K 31/4545; A61K 9/0014; A61K 9/006; A61K 9/06; A61K 9/08; A61K 31/135; A61P 17/02

(57) ABSTRACT

Compositions and methods for treating cutaneous radiation injury (CRI), including radiation dermatitis, radiation proctopathy, or oral mucositis, employing one or more functional inhibitor of acid sphingomyelinase (FIASMA).

### FIELD OF THE INVENTION

This invention is directed to compounds, compositions, and methods for treating a cutaneous condition or disorder induced by radiation exposure.

### BACKGROUND OF THE INVENTION

Cutaneous Radiation Injury (CRI) is damage to skin or underlying tissue in animals, including mammals and humans, which is induced by, or results from, exposure to radiation from industrial or military-related sources, e.g., radioactive materials used in nuclear power plants or in nuclear weapons. Mild skin damage, e.g., sunburn from overexposure to the sun, is not generally considered to fall within the definition of CRI; however, the term CRI can include skin conditions, such as Radiation Dermatitis, or other conditions such as Radiation Proctopathy, Oral Mucositis, or Severe Oral Mucositis, each of which can be observed following medical radiation therapy administered to an area of the body for treating cancer or other conditions treatable using radiation.

Radiation therapy in the medical field has been used successfully in treating locally or regionally advanced cancer and can be employed as a sole treatment or adjunctive to chemotherapy or surgery. The radiation exposure in such treatments, however, may cause severe burns of the skin and surrounding tissue as well as permanent changes in pigmentation. As many as 95% of patients treated with radiation therapy for cancer will experience a skin reaction. Certain skin reactions due to radiation treatments administered to breast cancer patients, for example, are referred to as “radiation dermatitis.”

Head and neck cancer patients can suffer inflammatory responses in the mouth, causing conditions such as oral mucositis, due to damage caused to the epithelial cells lining the mouth following radiation treatments to treat the head and neck cancer. When ulcerative lesions form in the mouth due to radiation treatment, or if the patient becomes unable to swallow solid food, the condition is considered as “Severe Oral Mucositis,” or “SOM.”

Radiation therapy administered to the pelvic area can cause damage to rectal tissue. A variety of disorders resulting from radiation therapy targeted to the pelvic area may affect the anorectal region, and may involve one or more of inflammatory, ischemic, infectious, traumatic, or neoplastic pathologies. Symptoms of anorectal disease include anal or rectal pain, urgency to move the bowels, fecal incontinence, diarrhea, rectal bleeding, and difficulty with evacuation of the rectum. These conditions are referred to as “radiation proctitis,” more recently and more accurately referred to as “radiation proctopathy.”

In view of the prevalence of these conditions in cancer patients undergoing radiation therapy, several strategies and treatments have been proposed for patients suffering from radiation dermatitis, but not have shown great success. Aloe vera and topical Vitamin C have been tried without improvement in the results of irradiated breast tissue.

Other reports indicate that the prophylactic and ongoing use of topical corticosteroid or a dexpanthenol-containing emollient can ameliorate, but not prevent, radiation dermatitis. Other reports have shown that moist skin care with 3% urea lotion delays the occurrence and reduces the grade of acute skin reactions in percutaneously irradiated patients with head and neck tumors. Negative side-effects from corticosteroids are well documented and their use is preferably avoided. Topical corticosteroids have little to no effect on pigmentation changes.

Biafine and Lipiderm were shown to have no radioprotective effect, while significant dermato-cytoprotective effects were shown from the use of amifostine in a retrospective analysis.

Misoprostol, a prostaglandin E(1) analog, has been found to be an effective radioprotector in animal studies preventing oncogenic transformation of Syrian hamster embryos exposed to radiation in utero. However, successful results were not obtained in humans.

Attempts to prevent or treat radiation proctopathy or proctitis have also been largely unsuccessful. Many topical treatments used for anorectal or lower bowel conditions appear to be ineffective. For example, use of the anti-inflammatory agent 5-aminosalicylic acid (5-ASA), previously used to treat Inflammatory Bowel Disease (IBD), was unsuccessful in treating radiation proctopathy or proctitis, with and without a steroid such as hydrocortisone. Sucralfate had also been recommended for radiation proctopathy but has proven ineffective.

Topical therapies, such as nitroglycerin and calcium channel blockers, were also found ineffective for treating radiation proctopathy. Short chain fatty acid enemas have also been used to treat radiation proctopathy but are not readily available and are difficult to administer.

Recently, antioxidants, such as Vitamin A, Vitamin C, and Vitamin E formulated in a suppository have been described for treating radiation proctopathy or proctitis. Suppositories are solid dosage forms comprising medications for placement in the anus or vagina for the treatment of certain systemic conditions but can be used for localized treatment of anorectal and gynecologic disorders. For example, one common use of rectal suppositories is for the treatment of constipation.

Rectal suppositories are also used as an alternative form of drug delivery in patients that cannot receive medications by mouth, whereby the medication is absorbed by the mucus membrane and distributed systemically. Examples of these types of rectal suppositories include treatments for nausea and pain.

It has been proposed that damage to healthy tissue from radiation therapy may involve an endothelial response associated with signaling from the plasma membrane, via the acid sphingomyelinase/ceramide pathway (Corre, I., et al., Intl. J. Mol. Sci. (2013) 14, 22678-22696.) Protecting against endothelial damage, for example, by inhibiting acid sphingomyelinase (ASMase) activity, is suggested as a means for limiting radiation toxicity in normal tissues. Corre, supra, at 22685. Although inhibitors of ASMase are known, none have been developed for topical administration, nor demonstrated to be effective for prevention or treatment of CRI, generally, or radiation dermatitis, radiation proctopathy, or oral mucositis, specifically.

One class of ASMase inhibitors, known as tricyclic antidepressants, have not previously been known to be used for treating anorectal disorders such as radiation proctopathy or proctitis. Nor have dosage forms comprising a tricyclic antidepressant such as amitriptyline been formulated for anal or rectal administration or in situ (or non-systemic) treatment of radiation proctopathy or proctitis. Amitriptyline has been formulated in a suppository dosage form. However, amitriptyline suppositories were only prepared for systemic delivery and to treat depression—the known indication for amitriptyline. Another indication for amitriptyline suppositories for systemic delivery is insomnia, exploiting the sleep-inducing side effect observed for amitriptyline. Amitriptyline suppositories have not previously been known to be administered to patients undergoing, or that have undergone, pelvic radiation therapy or for treating radiation proctopathy or proctitis or any other in situ treatment within the lower intestinal tract.

More specifically, amitriptyline has not previously been formulated in a dosage form, such as a suppository or other anorectal delivery device for localized treatment of radiation proctopathy or proctitis. Nor has a tricyclic antidepressant, such as amitriptyline, been formulated as a controlled release formulation to bypass release within a low pH environment (acidic pH of less than 7), such as in the stomach, such that the drug release occurs primarily in the intestine, preferably the lower intestine for in situ delivery of the drug to treat radiation proctopathy or proctitis.

Another class of ASMase inhibitors, selective serotonin reuptake inhibitors (SSRI's) are not known to be available in suppository form; nor are SSRI's known to be previously used for treatment of radiation proctopathy or proctitis.

There are numerous anorectal diseases that may benefit from localized administration of TCAs, such as amitriptyline or SSRIs, such as sertraline. These conditions include (but are not limited to), inflammatory bowel disease (IBD) including ulcerative proctitis and Crohn's disease, anal fissures, internal hemorrhoids, radiation proctopathy, anal and rectal neoplasms, anal warts, anal dysplasia, solitary rectal ulcer syndrome, pruritis ani and anorectal ischemia. These conditions represent a variety of significant clinical problems for which limited treatment options are currently available.

ASMase inhibitors such as TCA's, e.g., amitriptyline, or other tricyclics, such as tricyclic antihistamines, e.g., loratadine or its metabolite desloratadine, or SSRI's, e.g., sertraline, are also not known to have been used for CRI conditions such as radiation dermatitis or oral mucositis. These drugs—tricyclic antidepressants and antihistamine and SSRI's—are typically provided as solid oral dosage forms. However, liquid oral dosage forms, such as oral solutions suspension, or emulsion (separately or collectively referred to herein as an “oral rinse” or, simply, a “rinse”) comprising a tricyclic active ingredient or an SSRI are not known to have been formulated for treating oral mucositis.

Patients having reduced production of saliva due to radiation damage to the salivary glands, and having difficulty swallowing an oral dosage form or orally dissolving rapid-disintegrating solid oral dosage form, can benefit from an oral solution, suspension, or emulsion which can be used as a rinse or mouthwash to deliver the active ingredient to the damaged tissue within the mouth and oral cavity.

The continued and increased use of radiation therapy in the treatment of cancer, and lack of available preventive or therapeutic medications for CRI, including radiation dermatitis, or radiation proctopathy, is a longstanding unmet patient need affecting millions of patients.

### BRIEF DESCRIPTION OF THE INVENTION

The subject invention concerns a method for ameliorating or reducing the severity of, preventing, reducing the incidence or recurrence of, inhibiting, treating, or reversing, Cutaneous Radiation Injury (CRI), resulting from environmental exposure to radiation for industrial or military radioactive sources, or radiation dermatitis, radiation proctopathy, or oral mucositis (including Severe Oral Mucositis, or SOM) resulting from therapeutic radiation procedures in cancer treatment. The term, CRI, generally includes radiation damage to the outer layer of the skin, or dermis, but may include damage to deeper skin layers, such as the subdermal layer or adipose layer. Radiation dermatitis, radiation proctopathy, and oral mucositis fall within the definition of CRI, but are generally considered to be conditions relating to damage of the dermis.

A method according to the invention comprises topically administering or applying to the skin surface or the mucous membrane of a patient suffering from CRI, including radiation dermatitis, radiation proctopathy, or oral mucositis, a composition comprising at least one acid sphingomyelinase (ASMase) inhibitor. A functional inhibitor of acid sphingomyelinase, known in the art by the acronym, FIASMA, includes certain selective serotonin reuptake inhibitors (SSRIs), tricyclic antidepressants (TCAs), tricyclic antihistamines, antiarrhythmics, adrenergic receptor blockers (ARBs), beta blockers, and the like.

SSRIs useful in the method of the invention include one or more of: citalopram, escitalopram, fluoxetine, fluvoxamine, paroxetine, or sertraline. A preferred SSRI is sertraline. According to a preferred embodiment, the dosage form of the invention comprises an ASMase inhibitor, such as an SSRI, at a dosage of between 0.1 mg-1000 mg; more preferably a dose of about 1 mg-500 mg, and most preferably a dose of about 5 mg-100 mg. It is contemplated that a preferred dose is about 10-100 mg of the SSRI, formulated in a 0.5-10% (w/w) composition. A topical composition typically comprises 0.5%-5% of at least one FIASMA, preferably about 1% to about 4% of at least one FIASMA, more preferably about 1% to about 3% of at least one FIASMA, and most preferably about 1.5% to about 2% of at least one FIASMA.

The dose can vary, being lower in dosage forms that more rapidly deliver drug to the site, or higher in dosage forms that slowly release drug.

Tricyclic antidepressants (TCAs) useful in the method or composition of the invention include one or more of: amineptine, amitriptyline, amoxapine, butriptyline, clomipramine, desipramine, dibenzepin, dosulepin, doxepin (in combination but not alone), imipramine, iprindole, lofepramine, maprotiline, norclomipramine, northiaden, nortriptyline, opipramol, protriptyline, tianeptine, or trimipramine. A preferred TCA is amitriptyline. According to one preferred embodiment, the dosage form of the invention comprises amitriptyline at a dosage of between 0.1-1000 mg; more preferably a dose of about 1 mg-100 mg, and most preferably a dose of about 5 mg-50 mg. A preferred dose is about 10-50 mg of the TCA, formulated in a 1-5% (w/w) composition. The dose can vary, being lower in dosage forms that more rapidly deliver drug to the site, or higher in dosage forms that slowly release drug.

More specifically, the method of the invention comprises the steps of:
providing a topical composition comprising a pharmaceutically effective amount of at least one FIASMA as an active ingredient, or a combination of two or more FIASMA drugs,
topically administering or applying an effective amount of the composition to a patient in need thereof prior to radiation therapy or following radiation therapy or both.

In a method for preventing, reducing the incidence or recurrence of, inhibiting, treating, ameliorating, or reducing the severity of, or reversing radiation proctopathy, the method can comprise the step of:
anorectally administering to a patient in need thereof, such as a patient undergoing radiation treatment of the pelvic area or a patient suffering from radiation proctopathy or proctitis, a dosage form comprising a pharmaceutically effective amount of one or more active ingredients, wherein at least one active ingredient is a FIASMA.

In a method for preventing, reducing the incidence or recurrence of, inhibiting, treating, ameliorating, or reducing the severity of, or reversing oral mucositis or severe oral mucositis, the method can comprise the step of:
Providing a solid oral dosage form comprising a FIASMA as an active ingredient, and
administering to a patient in need thereof, such as a patient undergoing radiation treatment for head and neck cancer, a solid oral dosage form comprising a pharmaceutically effective amount of one or more active ingredients, wherein at least one active ingredient is a FIASMA.

In one embodiment, the solid oral dosage form is a modified release dosage form for sublingual administration comprising FIASMA as an active ingredient.

In another method for preventing, reducing the incidence or recurrence of, inhibiting, treating, ameliorating, or reducing the severity of, or reversing oral mucositis or severe oral mucositis, the method can comprise the step of:
providing a liquid oral dosage form or “rinse,” as a solution, suspension, or emulsion, comprising one or more FIASMA as an active ingredient, and
administering to a patient in need thereof, such as a patient undergoing radiation treatment for head and neck cancer, the liquid oral dosage form comprising a pharmaceutically effective amount of the one or more FIASMA as an active ingredient in the liquid dosage form.

Controlled release dosage forms for sublingual administration are known in the art and can slow the dissolution of the dosage form so that release of the drug is extended over time, retaining presence, or increasing residence time of the active drug in the oral cavity. Such controlled release dosage forms can be a tablet, capsule, lozenge, pastille, troche, or thin film strip or the like as recognized be persons of ordinary sill in the pharmaceutical arts. Controlled release dosage forms for sublingual administration are also known to rapidly dissolve or disintegrate within the oral cavity to provide immediate administration of the active ingredient from the dosage form. Therefore “controlled release” is not limited to slowing dissolution but can be “controlled” in a manner to rapidly disintegrate in the mouth and dissolve faster than a standard rate of dissolution for “immediate-release” solid dosage forms, where “immediate release” generally refers to the release in the stomach without significant delivery of active prior to swallowing of the dosage form.

One embodiment of a method according to the invention comprises topically applying or topically administering a composition comprising an effective amount of at least one FIASMA. It would be understood that applying or administering the composition prior to radiation therapy can include retention of the composition at the location before, during, or after, the radiation therapy session. The method can be repeated for each radiation therapy session and can include administering or applying the composition at least one time per week, several times per week, daily, or multiple times daily, before, after, or between radiation therapy sessions. The method according to the subject invention can be continued after radiation treatment is completed, for example, for 1-5 days following radiation treatment, for one to three weeks following radiation treatment, or for one or more months following radiation treatment. Typically, the method comprises administering the composition from about one week to about one month after the radiation therapy sessions are discontinued.

Another method according to the subject invention can comprise orally administering a composition comprising an effective amount of at least one FIASMA. Such oral composition can be an immediate release or controlled release solid dosage form. For conditions affecting the oral cavity, such as oral mucositis or severe oral mucositis, oral dosage forms that dissolve in the oral cavity, and are not swallowed whole, can be preferred.

In certain patients suffering from damage to the salivary glands, and who fail to produce adequate saliva to dissolve a solid dosage form in the oral cavity, a liquid oral dosage form comprising a FIASMA as an active ingredient can be a preferred dosage form. Such liquid oral dosage forms are known as “rinses,” “oral rinses,” “washes,” “mouthwashes,” or can be used as “sprays.” A preferred term used in this disclosure for a liquid oral dosage form is “rinse.” These liquid oral dosage forms would be understood to be in the form of a liquid solution, emulsion, or suspension, and can include thickeners or gelling agents for increasing the viscosity of the solution, emulsion, or suspension.

In a patient undergoing radiation therapy in the pelvic area, radiation proctopathy, a preferred method can comprise providing a solid suppository or rectal plug dosage form comprising a composition comprising a FIASMA and placing the dosage form within the rectal cavity for a period of time required for delivery of the active ingredient to the site.

The subject invention also concerns a topical pharmaceutical composition comprising as an active drug or active substance, at least one functional inhibitor of acid sphingomyelinase (FIASMA), useful for preventing, reducing the incidence or recurrence of, treating, ameliorating, or reducing the severity of, or reversing, Cutaneous Radiation Injury (CRI), including radiation dermatitis or radiation proctopathy.

The invention can also include a suppository dosage form for use in preventing, reducing the incidence or recurrence of, treating, ameliorating, or reducing the severity of, or reversing, radiation proctopathy.

Dosage forms of the subject invention can comprise a soluble base and a TCA, e.g., amitriptyline, a tricyclic antihistamine, such as loratadine, an SSRI, such as sertraline, or a combination of one or more TCA, tricyclic antihistamine, or SSRI, and can further include other pharmaceutically acceptable excipients as conventionally employed in pharmaceutically acceptable dosage forms.

In the method employing a suppository or a rectal plug delivery device for delivery of the FIASMA to the anorectal cavity and in situ administration of the drug, the dosage form can be provided with or incorporated into the rectal plug delivery device as a controlled release formulation, having one or more excipient that can retard the release of active drug incorporated into the dosage form or which is coated onto the outer surface of the dosage form or a particle, granule or bead within the dosage form comprising the active ingredient. A controlled release dosage form can also be provided for oral administration wherein the oral dosage form is formulated as a delayed release dosage form which bypasses the acidic environment in the stomach and releases active ingredient in intestinal tract where the pH is above 7.0. Such controlled release dosage forms can be formulated using an enteric coating on a tablet, provided within delayed release capsule or caplet, formulated in a slow release matrix composition, or a combination of the above.

Where the dosage form is a rectal plug delivery device, the device can be an insoluble plug forming a housing which has an internal chamber for containing therewithin a composition comprising the FIASMA, which can be a TCA, an SSRI, or various combinations thereof. For example, the chamber of the rectal plug can be filled with a viscous composition comprising a TCA or can contain a suppository dosage form comprising the TCA active ingredient.

An alternative embodiment of a rectal plug delivery device useful for carrying out the method of the invention comprises a porous compressible foam material infused or coated with a composition comprising the TCA, e.g., amitriptyline, the SSRI, e.g., sertraline, or combinations thereof. The devices described for the method of the invention can contain an effective dose of the FIASMA or FIASMA combination between about 0.1 mg to about 1000 mg or greater.

A composition in accordance with the invention can also include two or more active pharmaceutical ingredients formulated or mixed together in a single fixed-dose composition, such as a composition comprising two or more different TCAs, two or more different SSRIs, or one or more TCA in combination with one or more SSRI or the like.

A composition for use in a method of the invention can comprise between about 0.1-1000 milligrams (mg) of one or more FIASMA. A topical composition useful in accordance with a method of the invention can be provided as a topical liquid, but is preferably formulated as a topical cream, gel, lotion, or ointment. One preferred FIASMA useful as an active ingredient in a composition and method of the invention is the SSRI, sertraline. Another FIASMA useful as an active ingredient in a composition and method of the invention is the TCA, amitriptyline. Yet another FIASMA useful as an active ingredient in a composition and method of the invention is the tricyclic antihistamine, loratadine, or its metabolite, desloratadine. Other FIASMA compounds may be substituted or can be provided in combination in a composition or method of the invention.

The topical dosage form can comprise a conventional and commercially available pharmaceutical base composition pharmaceutically compatible with the one or more FIASMA used as the one or more active ingredients, and can further include other pharmaceutically acceptable excipients as conventionally employed in topical or oral dosage forms. These commercially available pharmaceutical bases can serve as a vehicle or medium for the active drug substance and can comprise one or more of an emollient, penetration enhancer, solvent, gelling agent, thickening agent or viscosity-enhancing agent, preservative, or other suitable excipient commonly used in topical preparations.

Preferably, the FIASMA for topical administration is provided in an immediate-release topical formulation which allows targeted dosing and immediate delivery of the drug from the composition to the skin upon application or administration of the composition. Retention of the FIASMA component at the site of administration or application can be preferred. Accordingly, a composition of the invention may exclude a penetration enhancer to maintain the composition, and the active substance, on the surface of the skin for a longer period of time relative to a composition comprising a penetration enhancer which allows or facilitates penetration of the active substance into or below the skin where it can be absorbed systemically. Alternatively, in certain instances, a penetration enhancer may be preferred and employed within the composition.

An oral dosage form of a composition can be immediate-release or may be formulated with excipients, such as polymers, gels, gums, waxes, or the like, or incorporated into a matrix or comprising a coating to slow, delay, sustain, or extend release of the active ingredient from the composition following oral administration. For example, a solid dosage form for sublingual administration of the FIASMA in treating oral mucositis can be formulated for dissolving within the oral cavity, and can include excipients which control, delay, slow, sustain or extend the release of the drug from the dosage form. Alternatively, the solid oral dosage form can be formulated to rapidly dissolve or disintegrate within the oral cavity, for example, when treating oral mucositis. One preferred dosage form for treating oral mucositis is a liquid oral solution, emulsion, or suspension comprising a FIASMA, and is preferably formulated as a rinse comprising an SSRI, such as sertraline.

According to a further aspect of the invention, the dosage form can include a second active, such as a TCA or SSRI ingredient, or can comprise a second active which is not a TCA or SSRI, for example, an active ingredient which falls within a different class of drug, i.e., other than a tricyclic or SSRI. In a preferred embodiment, a composition comprises a first active ingredient which is a TCA and a second active ingredient which is a different TCA or is an SSRI or other class of compound which is not a TCA. For example, a second active ingredient can be an antioxidant, such as Vitamin A, Vitamin C, Vitamin D, or Vitamin E. Preferably, the second active in the composition is not an anti-inflammatory agent such as a non-steroidal anti-inflammatory drug (NSAID) nor an anesthetic agent, such as a local anesthetic, e.g., lidocaine.

In another preferred embodiment, a composition of the invention comprises a first active ingredient which is an SSRI and a second active ingredient which is a different SSRI, or is a TCA or other class of compound which is not an SSRI. For example, a second active ingredient can be an antioxidant, such as Vitamin A, Vitamin C, Vitamin D, or Vitamin E. Preferably, the second active in the composition is not an anti-inflammatory agent such as a non-steroidal anti-inflammatory drug (NSAID) nor an anesthetic agent, such as a local anesthetic, e.g., lidocaine.

Preferably, the method is carried out using a FIASMA formulated in a topical composition which can deliver an effective dose to a target area of the skin which is directly or collaterally exposed to radiation during radiation therapy or treatment. For example, the active ingredient can be formulated as a liquid composition and administered as drops to the target area of the skin. Alternatively, the FIASMA can be formulated in a composition comprising a thickening agent or viscosity-enhancing agent as a lotion, cream, ointment, or gel for topical application and delivery of the FIASMA to the target skin area of the patient. The topical formulation can be rubbed onto or into the target area of the skin prior to or following radiation therapy.

In the method employing a solid oral dosage form, the active ingredient can be provided as a particle, granule, bead, or other conventional form and manufactured in a dosage form which is a tablet, capsule, caplet, or the like, as would be readily understood in the art. The oral dosage form can be provided, for example, as a controlled release dosage form where the active ingredient is formulated to delay release from the final dosage form following administration, such as an enteric coated tablet, delayed release capsule or caplet, or the active ingredient or ingredients can be formulated to slowly release active ingredient from the dosage form following administration, such as in matrix composition.

In the method employing a liquid oral dosage form, the active ingredient can be dissolved, emulsified, or suspended within a compatible and pharmaceutically acceptable carrier. Excipients or other inactive ingredients can be included for providing color, flavor, viscosity, and pleasant mouth-feel for the patient to facilitate compliance. Combinations of these dosage forms can also be employed. A second active ingredient which is not a FIASMA can also be included. For example, a local anesthetic can be included which is useful for reducing pain during and after the treatment or an antiseptic, other anti-inflammatory, or antimicrobial can also be included.

Thus, in accordance with the invention, the method for preventing, reducing the incidence or recurrence of, inhibiting, treating, ameliorating or reducing the severity of, or reversing, cutaneous radiation injury (CRI), comprises topically administering to a target area of skin of a patient in need thereof at least one time per day prior to, during, and after radiation treatment, 0.5-50 ml or more, depending on the size of the area to be treated, a composition comprising an effective amount, e.g., from about 0.1% to about 10% w/w, of a FIASMA, such as a TCA (e.g., amitriptyline), tricyclic antihistamine (e.g., loratadine), an SSRI (e.g., sertraline), or a combination of two or more FIASMA's.

The method for preventing, treating, or ameliorating radiation dermatitis also comprises topically administering to a target area of skin of a patient in need thereof at least one time per day prior to, during, and after radiation treatment, 0.5-50 ml or more, depending on the size of the area to be treated, a composition comprising an effective amount, e.g., from about 0.5% to about 10% w/w, of a FIASMA, such as a TCA (e.g., amitriptyline), tricyclic antihistamine (e.g., loratadine), an SSRI (e.g., sertraline), or a combination of two or more FIASMA's. The method also includes preventing, treating, or ameliorating radiation proctopathy, by topically administering or delivering to a target area a patient in need thereof at least one time per day prior to, during, and after radiation treatment, 0.5-50 ml or more, depending on the size of the area to be treated, a composition comprising an effective amount, e.g., from about 0.5% to about 5% w/w of a FIASMA, such as a TCA (e.g., amitriptyline) or an SSRI (e.g., sertraline.)

The method for preventing, treating, or ameliorating oral mucositis can comprise orally administering to a target area of skin or mucous membrane in the oral cavity of a patient in need thereof at least one time per day prior to, during, and after radiation treatment, 0.5-50 ml or more, depending on the size of the area to be treated, a composition comprising an effective amount, e.g., from about 0.5% to about 10% w/w, of a FIASMA, such as a TCA (e.g., amitriptyline) or an SSRI (e.g., sertraline. It would be understood that the term “oral mucositis” includes “Severe Oral Mucositis.”

A preferred treatment of oral mucositis can employ a dosage form comprising a slow dissolving adhesive tablet, such as a mucoadhesive tablet, an oral mouth rinse, or oral thin wafer or film, wherein the dosage form is disposed within the mouth, e.g., sublingually or buccally, wherein the dosage form is formulated in a way to release the active ASMase inhibitor, preferably about 1-50 mg sertraline, over a period of about 15 minutes up to an hour for release and delivery of the active for protecting against oral mucositis, including severe oral mucositis (SOM).

One embodiment of an oral dosage form used in treating oral mucositis or SOM comprises a lozenge or troche which is retained in the mouth to slowly dissolve and thereby slowly release active ingredient from the dosage for to provide local or in situ protection against CRI that can result from radiation therapy administered to a patient suffering from head and neck cancer. It would be understood that the lozenge or troche can be formed to include a stick or stem useful as a handle for inserting and removing the dosage form, akin to a candy-on-a stick (a “lollipop”) or other long-lasting confectionary.

Such “lollipop” systems for delivering a pharmaceutical ingredient to buccal mucosa are described. For example, U.S. Pat. No. 6,103,257 describes a hard candy matrix mounted on a handle to form a lollipop, a method of manufacturing the hard candy and formation of the lollipop embodiment, and a method for delivering local anesthetics to the buccal mucosa using the hard candy or lollipop embodiment. The disclosure of this U.S. Pat. No. 6,103,257 is hereby incorporated by reference in its entirety into this specification.

A topical composition employed in the method can comprise 5-100 mg amitriptyline per ml of the composition or can comprise 1-100 mg sertraline per ml of the composition or can comprise 10-90 mg amitriptyline per ml of the composition and 1-50 mg sertraline per ml of the composition. Amitriptyline is preferred to be used at less than 40 mg per ml of the composition and more preferably about 20-30 mg per ml of the composition.

The above topical compositions can also include an antioxidant such as Vitamin A, Vitamin C, Vitamin D, and Vitamin E.

Preferably, the composition used in the method of the invention is formulated as a topical dosage form which delivers the effective dose of active ingredient in situ to the target area of the patient. This can be carried out using a composition formulated with a pharmaceutically acceptable base to form a lotion, cream, ointment, or gel for topical delivery of the active ingredient to a target area of skin of the patient. For treating oral mucositis, the composition can be formulated as an oral solution, emulsion, or suspension, referred to herein as a “rinse.”

Alternatively, the composition can comprise amitriptyline and a second tricyclic antidepressant which is not amitriptyline, such as amineptine, amitriptyline, amoxapine, butriptyline, clomipramine, desipramine, dibenzepin, dosulepin, doxepin (in combination but not alone), imipramine, iprindole, lofepramine, maprotiline, norclomipramine, northiaden, nortriptyline, opipramol, protriptyline, tianeptine, or trimipramine.

The composition comprising sertraline can comprise a second selective serotonin reuptake inhibitor which is not sertraline, such as citalopram, escitalopram, fluoxetine, fluvoxamine, or paroxetine.

The pharmaceutical composition of the invention can be provided as a topical pharmaceutical composition or can be an oral dosage form-either solid or liquid-comprising an effective amount of the one or more active ingredient, such as amitriptyline, sertraline, or a combination thereof for delivery of the one or more active ingredients to the patient. In a preferred embodiment, a topical composition of the invention is free of, does not include, or excludes, an anti-inflammatory agent and an anesthetic agent.

A preferred topical pharmaceutical composition comprises 10-20 mg amitriptyline per ml of the composition, or 1-100 mg sertraline per ml of the composition, or 15-20 mg amitriptyline per ml of the composition and 1-10 mg sertraline per ml of the composition. Any of the above can further comprise an effective amount of an antioxidant such as Vitamin A, Vitamin C, Vitamin D, or Vitamin E.

An oral dosage form of the invention can be formulated as a controlled release oral dosage form, which is either a fast-dissolving oral dosage form or a slow or delayed-release dosage form.

It would be understood that the method and compositions and dosage forms of the invention can be useful for preventing, treating, or ameliorating other dermal conditions such as atopic dermatitis, thermal burn, sunburn, dermatomyo-fibromas, exposure-induced wrinkles, or the like.

### BRIEF DESCRIPTION OF THE DRAWINGS

FIG. 1 shows the results upon examination of skin injury after 30 Gy focal irradiation. Panel A of FIG. 1 is a summary of the skin injury scores for individual mice up to 4 weeks after 30 Gy. Panel B is graphical representation of the observed changes in the radiation skin injury score over time. Data are presented as mean +/−SEM. N=5 mice per treatment group.

FIG. 2 shows the results of examination of skin injury after 30 Gy focal irradiation. Panel A of FIG. 2 is a summary of skin injury scores for mice treated with 4% sertraline 3 times before irradiation, 4% sertraline 2 times after irradiation or placebo 5 times before and after irradiation. Skin injury scores were examined every week for up to 6 weeks after irradiation. Panels B-D of FIG. 2 illustrate comparisons of skin injury scores over time between cohorts of mice that received different treatments. Data are presented as mean +/−SEM. N=5 mice per treatment group. *P<0.05 by two-way ANOVA with Bonferroni post-hoc test.

FIG. 3 shows the percentage of patients—treated with either RAD-100 or an identical vehicle-during and after radiation treatment, who score an RTOG score of 2 or higher.

FIG. 4 shows the effect of 1% sertraline on radiation-induced oral mucositis in mice.

### DETAILED DESCRIPTION OF THE INVENTION

The present invention provides an innovative method for preventing, reducing the incidence or recurrence of, inhibiting, treating, ameliorating, or reducing the severity of, or reversing a skin condition in a mammal, including humans, and can employ the use of a novel composition or dosage form to carry out the method of treatment. The methods and compositions of the invention are useful for treating Cutaneous Radiation Injury, or CRI.

Hereinafter, the terms “treat,” “treating,” or “treatment of” in relation to Cutaneous Radiation Injury, or “CRI,” or a specific condition considered a type of CRI, can mean or refer to, or can be substituted by, any one of “preventing,” “reducing the incidence of” “reducing the recurrence of,” “inhibiting,” “ameliorating,” “reducing the severity of,” “reversing” or “treating” CRI. The specific term, “preventing,” “reducing the incidence of” “reducing the recurrence of,” “inhibiting,” “ameliorating,” “reducing the severity of,” or “reversing” CRI is used where it is intended to designate that treatment only, or a term may be expressly excluded from “treatment” when intended not to mean that term. For example, a method for “reducing the incidence of” may be used to mean only reducing the incidence of CRI and not “inhibiting” CRI. Alternatively, “a method for treating CRI, excluding reducing the occurrence of CRI” can mean a method for preventing, reducing the incidence of, inhibiting, ameliorating, reducing the severity of, or reversing CRI.

The term “Cutaneous Radiation Injury,” or “CRI” is accepted in the medical field as being defined as damage to skin or underlying tissue in animals, including mammals and humans, which is induced by, or results from, exposure to radiation from industrial or military-related sources, e.g., radioactive materials used in nuclear power plants or in nuclear weapons, or in medical radiation therapy. Therefore, the term CRI can include skin conditions such as Radiation Dermatitis or Radiation Proctopathy, observed following medical radiation therapy. Mild skin damage, e.g., sunburn from overexposure to the sun, is not generally considered to fall within the definition of CRI.

“Radiation dermatitis” refers to a disorder of the skin resulting from radiation therapy of an area of the body. For example, radiation therapy is a known and accepted treatment for cancerous breast tissue in women and men. Inflammation of the skin covering the targeted cancerous tissue, and exposed to the radiation, can result in damage to the skin. These signs and symptoms are known as radiation dermatitis. The term is used herein as it is clinically understood by dermatologists and other physicians that treat such disorders.

“Radiation proctopathy” or its synonym, “radiation proctitis,” refers to a disorder of the rectum causing alteration of rectal function, including inflammation of the lining of the rectum, resulting from radiation therapy of the pelvic area. Radiation therapy of the pelvic area is a known and accepted treatment procedure for cancer of urogenital tissue, for example, treating prostate cancer in men, or treating cervical cancer in women. The term is used herein as it is clinically understood by physicians that treat such disorders.

“Oral mucositis” refers to a condition that results from radiation therapy used in treating head and neck cancers. A more severe condition aptly termed “Severe Oral Mucositis” or “SOM” can often be associated with ulcerative tissue in the mouth or when patients cannot swallow food or drink. Oral mucositis is a common, debilitating complication of cancer treatments, particularly radiation. It can lead to several problems, including pain, nutritional problems as a result of inability to eat, and increased risk of infection due to open sores in the oral mucosa. Use of the term “oral mucositis” herein includes “Severe Oral Mucositis,” and these terms can be used interchangeably.

“Emulsion” is a general term encompassing a macroemulsion, microemulsion, or nanoemulsion. A macroemulsion is a kinetically stable dispersions of immiscible liquids. A microemulsion is a kinetically stable as well as thermodynamically stable dispersion of immiscible liquids having a droplet size in the micrometer size range. A nanoemulsion is a kinetically stable and thermodynamically stable dispersions of immiscible liquids having a droplet size ranging from about 20 nm to about 200 nm.

One embodiment of the invention concerns a method for treating Cutaneous Radiation Injury (CRI) by administering an effective amount or dose of a Functional Inhibitor of Acid Sphingomyelinase (FIASMA). Examples of FIASMAs include certain selective serotonin reuptake inhibitors (SSRIs), tricyclic antidepressants (TCAs), antihistamines (preferably tricyclic antihistamines such as loratadine or its metabolite desloratadine, antiarrhythmics, antipsychotics, antidiarrheals, adrenergic receptor blockers (ARBs), beta blockers, Estrogen Receptor Modulators, and the like.

Examples of SSRIs useful in accordance with the subject invention are citalopram, escitalopram, fluoxetine, fluvoxamine, paroxetine, and sertraline. Preferred SSRIs useful in accordance with the subject invention include fluoxetine, fluvoxamine, paroxetine, and sertraline.

Examples of TCAs useful in accordance with the subject invention are amineptine, amitriptyline, amoxapine, butriptyline, clomipramine, desipramine, dibenzepin, dosulepin, doxepin (in combination but not alone), imipramine, iprindole, lofepramine, maprotiline, norclomipramine, northiaden, nortriptyline, opipramol, protriptyline, tianeptine, and trimipramine. Preferred TCAs useful in accordance with the subject invention are amitriptyline, desipramine, and imipramine.

Examples of a tricyclic antihistamine useful in accordance with the subject invention includes promethazine, cyproheptadine, latrepiridine, rupatadine, loratadine, and desloratadine. A preferred tricyclic antihistamine useful in accordance with the subject invention is loratadine.

The invention includes the use of at least one FIASMA, such as a TCA, tricyclic antihistamine, or an SSRI and can include the use of at least two different FIASMAs from the same class of drug or at least two different FIASMAs from different classes of drug. For example, the subject invention can include treatment of CRI using at least two TCAs, at least one TCA and at least one SSRI or tricyclic antihistamine, or at least one of a TCA, tricyclic antihistamine, or SSRI, and at least one additional active ingredient which is not a TCA, tricyclic antihistamine or SSRI.

A preferred TCA for use in accordance with the subject invention is amitriptyline. Amitriptyline, marketed in the United States under the brand name ELAVIL® (AstraZeneca PLC, Cambridge England) is primarily used to treat a number of mental illnesses, including major depressive disorder, anxiety disorders, and less commonly attention deficit hyperactivity disorder (ADHD) and bipolar disorder. Other uses include prevention of migraines, treatment of neuropathic pain such as fibromyalgia and postherpetic neuralgia, and less commonly insomnia.

A preferred SSRI for use in accordance with the subject invention is sertraline. Sertraline, marketed in the United States under the brand name Zoloft® (Pfizer, New York, NY USA) is primarily used to treat depression, panic attacks, obsessive compulsive disorder, post-traumatic stress disorder, social anxiety disorder (social phobia), and a severe form of premenstrual syndrome (premenstrual dysphoric disorder).

A preferred tricyclic antihistamine useful in accordance with the subject invention is loratadine (the active ingredient in the antihistamine product, Claritin), or the loratadine metabolite, desloratadine (the active ingredient in the antihistamine product Clarinex.)

Amitriptyline, loratadine, and sertraline have not previously been known to be useful for prevention or treatment of Cutaneous Radiation Injury (CRI), which includes radiation dermatitis.

In a preferred embodiment, at least one FIASMA can be incorporated into a topical pharmaceutical composition for administration by placement or application onto the surface of the skin or a mucous membrane for the prevention, amelioration, or treatment of a dermal condition or disorder, such as radiation dermatitis or oral mucositis. This composition can be administered once daily, or more or less frequently as prescribed by the patient's physician or healthcare provider.

Topical compositions or preparations as dosage forms are well known in the art and are used conventionally in medical treatments. A preferred embodiment in accordance with the subject invention is to incorporate a FIASMA into a pharmaceutically compatible base composition to make a topical lotion, cream, gel, or ointment comprising the at least one FIASMA as an active pharmaceutical ingredient (API) or agent. Another embodiment of the invention includes incorporating at least two different FIASMAs, e.g., a combination of at least one TCA and at least one SSRI, as active agents into a pharmaceutically compatible base composition to make a topical lotion, cream, gel, or ointment. A preferred embodiment comprises the one or more active ingredients thoroughly mixed into a pharmaceutically acceptable cream or lotion base to provide a homogeneous mixture. For active ingredients that incompletely solubilize in the base, a suspension can be formed, wherein the suspension comprises an active ingredient which is thoroughly mixed to evenly disperse the active throughout the base. The composition of the invention can include other pharmaceutically acceptable excipients as conventionally employed in topical dosage forms. For example, stabilizers, emulsifiers, penetration enhancers can be included as desired and in accordance with conventional topical pharmaceutical formulation practices.

In another embodiment of the invention, the active ingredient, such as one or more TCA, tricyclic antihistamine, or SSRI can be formulated for oral administration. In one embodiment, the oral dose is a solid oral dosage form provided as an immediate release dosage form. In one embodiment, the solid oral dosage form is provided as controlled release dosage form. Yet another oral dosage form is a liquid oral dosage form comprising an effective amount of a FIASMA, such as the SSRI, sertraline. It has been identified that the tricyclic antihistamine can provide a preferred taste profile or mouth-feel for oral dosage forms used for in situ oral or mouth treatment. These liquid oral dosage forms are known as “rinses,” “washes,” “mouthwashes,” or “sprays.” A preferred term used in this disclosure for liquid oral dosage form is “rinse.” These liquid oral dosage forms would be understood to be in the form of a liquid solution, emulsion, or suspension, and can include thickeners or gelling agents for increasing the viscosity of the solution, emulsion, or suspension, and thereby increasing the retention or residence time of the composition at the target site.

In one embodiment, a solid oral dosage form is provided for sublingual or buccal administration of the invention includes a pharmaceutical composition in unit dosage form (e.g., a lozenge or troche, a pill, a tablet, a film, or a strip) comprising at least one FIASMA and in a preferred embodiment is prepared as a controlled release formulation. Regarding the size of the unit dosage form, e.g., lozenges, the simplicity and cost of production will also be taken into account, as well as the preferences of consumers. The size of the sublingual or buccal dosage form can vary from approximately 5 mm to 20 mm, or from approximately 5 mm to 18 mm, or from approximately 7 mm to 18 mm, or from approximately 10 mm to 15 mm. If the sublingual or buccal dosage form has a round or almost round shape, then the size range is a suitable diameter value. The thickness of the lozenges in most cases will be from 1 mm to 10 mm, and more generally from 3 mm to 5 mm.

A sublingual or buccal dosage form can be in the form of monolayer, bilayer, or multilayer. A sublingual or buccal dosage form having soluble mucoadhesive properties can be made by pressing powders, including mucoadhesive hydrocolloids. As an example, an adhesive pastil can be made by mixing dry powders, including gum arabic for adhesive properties and at least one FIASMA as an active ingredient. Residence time in the oral cavity for the sublingual or buccal dosage forms can be optimized according to the dissolution rate and total time of dissolution for the product. For example, a sublingual or buccal controlled release lozenge, mucoadhesive tablet, or film can be prepared to have a residence time in the oral cavity of at least one minute, preferably between one to 30 minutes, and more preferably 5-15 minutes after administration.

In one aspect of the invention, the sublingual or buccal dosage form is a mucoadhesive tablet. A mucoadhesive tablet for sublingual or buccal administration can be made by compressing at least one mucoadhesive polymer to impart mucoadhesion, and at least one FIASMA as an active ingredient. The mucoadhesive polymer may be one or more selected from the group consisting of cellulose-based polymers, gum-type polymers, nonionic polymers, anionic polymers, derivatives thereof, mixtures thereof, and the like. The cellulose-based polymer can be a hydroxypropyl cellulose, hydroxypropylmethyl cellulose, hydroxyethyl cellulose, hydroxypropylethyl cellulose, carboxymethyl cellulose, carboxypropyl cellulose, mixtures thereof, and the like. The gum-type polymer can be xanthan gum, carrageenan gum, Karayan gum, and the like. The nonionic polymer can be polyvinyl alcohol, polyvinylpyrrolidone, polyvinylpyrrolidone, polyvinylpyrrolidone-vinylacetate copolymer, polyethylene oxide, and the like. The anionic polymer can be polyacrylic acid, Carbopol® polymer and its neutralized polymers, other polyalkylvinylether-maleic acid copolymer (Trade name, Gantrez), polyalginic acid and salts thereof, chitosan, hyaluronic acid and derivatives thereof, and the like.

The mucoadhesive tablet can have a backing layer to delay the rate of disappearance of the formulation in the oral cavity by lowering the penetration rate of saliva. The backing layer can be composed of at least one water-insoluble polymer, at least one wax, or a combination thereof. Water-insoluble polymers include: polyvinylacetate; ethyl cellulose; propyl cellulose; polymethyl methacrylate; methacrylic acid copolymers such as methacryloylethylbetaine/methacrylate copolymer, methacrylic copolymer (methacrylic acid copolymers; Eudragit L 100, Eudragit L 12,5, Eudragit L 100-55, Eudragit L 30D-55), aminoalkyl methacrylate copolymers; Eudragit E 100, Eudragit E 12,5, Eudragit RL 100, Eudragit RL 30D), cellulose acetate phthalate, and mixtures thereof. Waxes can include: carnauba wax, yellow wax, white wax and the like. Additionally, any polymer that does not melt in the oral conditions between pH 6 to 8 can also be useful as the backing layer.

In another aspect, the invention features a pharmaceutical composition in unit dosage form formulated for oral administration, such as sublingual or buccal administration, the unit dosage form including from 5 to 100 mg of a FIASMA (e.g., a dosage form comprising 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, or greater, of sertraline.

In another aspect, the invention features a pharmaceutical composition in unit dosage form formulated for oral administration, such as sublingual or buccal administration, the unit dosage form including from 5 to 100 mg of a FIASMA (e.g., a dosage form comprising 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, or greater, of loratadine, or its metabolite, desloratadine. In one preferred embodiment, the dosage form for oral administration by buccal or sublingual administration can be provided as a lollipop, shaped as desired and including a paper or plastic stick or stem as a handle to facilitate insertion into and removal from the oral cavity.

Alternatively, the oral dosage form can be a liquid dosage form, such as an oral solution, emulsion, or suspension comprising about 1% to about 10% (w/w) of a FIASMA, e.g., an SSRI such as sertraline in a pharmaceutically acceptable and compatible carrier. A preferred oral solution comprises 10% sertraline. Controlled release oral dosage forms include delayed release formulations created by coating a solid dosage form with a polymeric film, which is insoluble in the acidic environment of the stomach, and soluble in the neutral environment of the small intestine.

Composition of the invention may include conventional additives or other excipients, such as plasticizers, pigments, colorants, stabilizing agents, glidants, and the like.

In one embodiment, the FIASMA is provided in an oral dosage form as a controlled-release formulation which allows delivery of the drug over a period of time as compared to an immediate-release formulation which provides delivery of all the drug from the dosage form as soon as it is administered. Controlled release formulations known in the art include the use of coatings, such as enteric-coated tablets, beads, or pellets, ion exchange resins, waxes, alginates, gelling agents, such as cellulosic hydrogels (e.g., hydroxypropyl methyl cellulose, or HPMC) or polymeric acrylamides (e.g., CARBOPOL®) formulated with an active agent, and a suitable vehicle.

The present invention provides a method for preventing, ameliorating, or treating CRI by administration of a FIASMA. The method includes a step of providing a composition comprising between about 0.001-100 grams of active ingredient and delivering the drug to a patient in need thereof, either directly to the skin as a topical composition, such as a cream or lotion or ointment, or formulated in an oral dosage form.

According to one preferred embodiment, the FIASMA contained in the topical dosage form is amitriptyline provided at a dosage of between 1-1000 mg; more preferably a dose of about 5 mg-150 mg, and most preferably a dose of about 10-100 mg, administered as about 0.5% to about 10% w/w topical composition. For example, a composition comprising 1% to 10% active ingredient (10-100 mg of active ingredient per 1 ml of the composition) can be administered in amounts of about 0.5 ml to about 5 ml per topical application or greater depending on the size of the area to be treated. Preferably, the composition is topically administered in about 1 ml to about 2 ml amounts to deliver 10-100 mg of the active ingredient to the skin for preventing or treating CRI.

In another preferred embodiment, the FIASMA contained in the topical dosage form is sertraline provided at a dosage of between 1-1000 mg; more preferably a dose of about 5 mg-150 mg, and most preferably a dose of about 10-100 mg, administered as about 0.1% to about 10% w/w topical composition. For example, a composition comprising 0.1% to 10% active ingredient (1-100 mg of active ingredient per 1 ml of the composition) can be administered in amounts of about 0.5 ml to about 5 ml per topical application or greater depending on the size of the area to be treated. Preferably, the composition is topically administered in about 1 ml to about 2 ml amounts to deliver 1-200 mg of the active ingredient to the skin for preventing or treating CRI.

One preferred dose is administering at least 1 ml of a composition comprising 1% of a FIASMA as an active ingredient according to the subject invention. Another preferred dose is administering at least 1 ml of a 2% w/w composition comprising a FIASMA active ingredient according to the subject invention. Yet another preferred dose is administering at least 1 ml of a 3% w/w composition comprising a FIASMA active ingredient according to the subject invention. Still another preferred dose is administering at least 1 ml of a 4% w/w composition comprising a FIASMA active ingredient according to the subject invention. A further preferred dose is administering at least 1 ml of a 5% w/w composition comprising a FIASMA active ingredient according to the subject invention. Preferred doses can include the administration of at least 1 ml of a 6%, a 7%, an 8%, a 9%, a 10%, or greater than 10% w/w composition comprising a FIASMA active ingredient according to the subject invention.

It would be understood that the concentration of drug can be provided at incremental increases of about 0.05%. As a non-limiting illustration, the FIASMA can be provided in a concentration of 1.05%, lollipop%, 1.15%, 1.2%, 1.25%, 1.3%, 1.35%, 1.4%, 1.45%, 1.5%, 1.55%, 1.6%, 1.65%, 1.7%, 1.75%, etc., up to 10%, as desired to obtain the intended effect without negative side-effects.

The dose contained within the dosage form can vary, being lower in dosage forms that more rapidly deliver drug to the site, or higher in dosage forms that slowly release drug to the site. Dose variation can also depend on the active ingredient or ingredients contained within the composition. For example, a composition comprising more than one FIASMA can include each active at less than 10%, such as 0.1%-9.9%, preferably about 0.5% to about 5% of each active, such that the final composition comprises about 1% to less than 10% total active ingredient in the composition. This can advantageously lower the dose of each active ingredient being administered to the patient while providing an equivalent or substantially equivalent preventive or therapeutic effect. It is also contemplated that a fixed dose combination product comprising at least one TCA and at least one SSRI can provide a synergistic effect, wherein the efficacy is greater than the expected additive effect from the respective active ingredients used alone.

Preferably, the FIASMA for topical administration is provided in an immediate-release topical formulation which allows targeted dosing and immediate delivery of the drug from the composition to the skin upon application or administration of the composition. Retention of the FIASMA component at the site of administration or application can be preferred. Accordingly, a composition of the invention can exclude a penetration enhancer to maintain the composition, and the active substance, on the surface of the skin for a longer period of time relative to a composition comprising a penetration enhancer which allows or facilitates penetration of the active substance into or below the skin where it can be absorbed systemically. Alternatively, in certain instances, a penetration enhancer may be preferred and employed within the composition.

In a further aspect of the invention, the dosage form can include a second active, such as a TCA, tricyclic antihistamine, or SSRI ingredient, or can comprise a second active which is not a TCA, tricyclic antihistamine, or SSRI, for example, the second active is an active ingredient in a different class of drug. In a preferred embodiment, a composition comprises a first active ingredient which is a TCA and a second active ingredient which is a different TCA, a tricyclic antihistamine, or an SSRI or other class of compound which is not a TCA. For example, a second active ingredient can be an antioxidant, such as Vitamin A, Vitamin C, Vitamin D, or Vitamin E. Preferably, the second active in the composition is not an anti-inflammatory agent such as a non-steroidal anti-inflammatory drug (NSAID) nor an anesthetic agent, such as a local anesthetic, e.g., lidocaine.

In another preferred embodiment, a composition of the invention comprises a first active ingredient which is an SSRI and a second active ingredient which is a different SSRI, or is a TCA, tricyclic antihistamine, or other class of compound which is not an SSRI. For example, a second active ingredient can be an antioxidant, such as Vitamin A, Vitamin C, Vitamin D, or Vitamin E. Preferably, the second active in the composition is not an anti-inflammatory agent such as a non-steroidal anti-inflammatory drug (NSAID) nor an anesthetic agent, such as a local anesthetic, e.g., lidocaine.

The topical dosage form can comprise a conventional and commercially available pharmaceutical base composition pharmaceutically compatible with the one or more FIASMA used as the one or more active ingredients and can further include other pharmaceutically acceptable excipients as conventionally employed in topical or oral dosage forms. These commercially available pharmaceutical bases can serve as a vehicle or medium for the active drug substance and can comprise one or more of an emollient, penetration enhancer, solvent, gelling agent, thickening agent or viscosity-enhancing agent, preservative, or other suitable excipient commonly used in topical preparations. In one embodiment, the commercially available pharmaceutical base is PENCream™, an oil-in-water vanishing base available from Humco™.

A topical composition comprising a FIASMA useful in accordance with the subject invention can be formulated as is conventionally known in the pharmaceutical arts and can comprise one or more additional ingredients or excipients, such as at least one preservative, at least one solvent, at least one thickener, at least one surfactant, at least one stiffening agent, at least one emulsifying agent, at least one antitack agent, mixtures thereof, or the like.

Preservatives for the topical composition can include Phenoxy-2 ethanol, Alcool benzylique Emprove, and the like. Solvents for the topical composition can include Glycerine, Propanediol-1,2, or the like. Thickeners for the topical composition can include Carpool ETD 2020, Xanthan gum, or the like. Surfactants for the topical composition can include Brij S721, SP-Brij S2-MBAL, or the like. Stiffening agents for the topical composition can include hydrogenated oil such as Kolliwax HCO, or the like. Emulsifying agents for the topical composition can include Miglyol 812 N, or the like. Antitack agents for the topical composition can include Q7-9120 Silicone Fluid, or the like.

The present invention provides a method for treating anorectal disorders by in situ administration of an active pharmaceutical ingredient of the invention, such as a TCA, e.g., amitriptyline, a tricyclic antihistamine, e.g., loratadine, an SSRI, e.g., sertraline, or combinations thereof. The method includes a step of providing a composition comprising between about 0.001-100 grams of the active ingredient, and delivering the drug directly to the anorectal area, either as a topical composition, such as a cream or ointment, or formulated in a suppository or rectal plug dosage form. According to a preferred embodiment, a TCA contained within the suppository or rectal plug dosage form is amitriptyline at a dosage of between 1-1000 milligrams (mg); more preferably a dose of about 5 mg-150 mg, and most preferably a dose of about 10-100 mg, where one preferred dosage is a suppository or other dosage form comprising about 50 mg. In another preferred embodiment, an SSRI contained within the suppository or rectal plug dosage form is sertraline at a dosage of between 1-1000 milligrams (mg); more preferably a dose of about 5 mg-150 mg, and most preferably a dose of about 10-100 mg, where one preferred dosage is a suppository or other dosage form comprising about 100 mg. The dose contained within the suppository or rectal plug can vary, being lower in dosage forms that more rapidly deliver drug to the site, or higher in dosage forms that slowly release drug to the site. In addition, where two or more active pharmaceutical ingredient are provided in a fixed dose combination suppository product, the dose of each of the active ingredients may be reduced within the dosage form and thereby advantageously lower the risk or incidence of side effects.

Preferably, the method comprises providing a solid suppository or rectal plug dosage form comprising a composition having a TCA or SSRI as an active ingredient and placing the dosage form within the rectal cavity for a period of time required for delivery of the active ingredient to the site. The composition provided within the rectal plug delivery device can be a suppository.

Alternatively, the drug can be delivered using a rectal delivery device such as a rectal plug containing an effective dose of active ingredient, such as a FIASMA, or a combination more than one FIASMA. In one embodiment of a rectal plug device according to the invention, the plug device comprises an insoluble porous housing forming a chamber for containing the active ingredient. The active ingredient can be provided as a soluble suppository disposed within a chamber portion formed within the porous rectal plug housing. The suppository is exposed to bodily fluids when administered to cause dissolution and in situ delivery of the active ingredient from the suppository. In a preferred embodiment, the suppository is formulated as a controlled release formulation. Alternatively, the active ingredient can be formulated as a viscous, controlled release gel, ointment, or cream for filling the chamber within the rectal plug housing.

Other types of rectal plugs are known and can be adapted for use in accordance with the subject invention. For example, rectal plugs formed of a porous sponge-like polymeric material, such as polyurethane foam, are known for use in fecal incontinence. Such rectal plugs are marketed under the name PERISTEENT™ Anal Plug (Coloplast Corp., North Minneapolis, MN, USA). The polyurethane foam can be infused with a solution comprising an effective amount of a FIASMA can be adsorbed onto the material for delivery when inserted into the rectal cavity. These commercially available rectal plugs generally are provided in a compressed configuration for ease of insertion and are wrapped with a water-soluble film which dissolves following insertion and can expand to an extended configuration. Such compression and expansion are not required for in situ delivery of drug in accordance with the subject invention.

The rectal plug delivery device can comprise a distal flange 102 to prevent the entirety of the rectal plug delivery device from being taken up into the rectal cavity, thereby holding the rectal plug device in place during administration of the active ingredient. The active ingredient, such as a TCA or SSRI, can be provided as a pharmaceutical composition which is housed within a cavity (not shown) formed within the elongate member. The proximal end of the elongate member 101 comprises pores 103 for allowing bodily fluids to enter the cavity formed within the elongate member, and to allowing egress of the active ingredient from the composition contained within the elongate member. The rectal plug delivery device can have an extension piece connected to the distal flange, such as a strap or string, to facilitate removal of the rectal plug delivery device, functioning similarly to removal of a tampon.

The composition for rectal administration can be formed as a suppository, which dissolves or melts when administered within the rectal cavity. Alternatively, the composition can be an amorphous, or unformed, composition comprising a gel, cream, or ointment. Preferably, the composition is provided as a controlled-release composition, preferably an extended-release composition, which releases the active ingredient from the composition over time.

In another embodiment, the dosage form comprises an anal plug comprising a soft, compressible, porous material (e.g., a foam rubber or polymeric sponge-like material) wherein the active ingredient is infiltrated or infused within, or coated on, the porous compressible plug material.

A rectal plug delivery device of the invention can comprise a soft, compressible, porous material in an expanded configuration. In this embodiment, the rectal plug delivery device comprises at its proximal end a drug delivery component which expands when inserted into the rectal cavity, and conforms to the shape of the rectal cavity. Connected to the drug delivery component is an extension piece, such as a strap or string, to facilitate removal of the rectal plug delivery device, functioning similarly to removal of a tampon.

The composition comprising active ingredient according to the invention can be infused or infiltrated into the porous material forming the delivery component, as a liquid or gel, cream, or ointment. The composition comprising the active ingredient can be provided as a controlled-release composition, preferably an extended-release composition, which releases the active ingredient over time.

A rectal plug delivery device comprising porous material can be provided in compressed form, for ease of insertion into the rectal cavity. The compressed drug delivery component can include a water-soluble wrapper surrounding the drug delivery component. Following insertion of the rectal plug delivery device embodiment into the rectal cavity, bodily fluids dissolve the wrapper and allow the drug delivery component to expand to its expanded configuration. An extension piece, such as a strap or string, can be provided to facilitate removal of the rectal plug delivery device.

In one preferred embodiment, the active ingredient is provided in a controlled-release formulation which allows delivery of the drug over a period of time as compared to an immediate-release formulation which provides delivery of all the drug from the dosage form as soon as it is administered. Controlled release formulations known in the art include the use of coatings, such as enteric-coated tablets, beads, or pellets, ion exchange resins, waxes, alginates, gelling agents, such as cellulosic hydrogels (e.g., hydroxypropyl methyl cellulose, or HPMC) or polymeric acrylamides (e.g., CARBOPOL®) formulated with an active agent, and a suitable vehicle which melts or dissolves in rectal fluids. Such formulations and compositions, as well as methods of manufacturing suppositories or other controlled-release compositions are well known in the art.

In a method according to the invention, treating a CRI such radiation dermatitis employs a composition comprising at least one FIASMA, such as a TCA, e.g., amitriptyline, and/or at least one second FIASMA, such as a tricyclic antihistamine, e.g., loratadine, or an SSRI, e.g., sertraline. The composition employed in the method of the invention can be provided in a topical dosage form. In addition, the subject invention comprises a method for treating, ameliorating, or preventing a skin disorder or condition such radiation dermatitis using an oral dosage form comprising at least one TCA such as amitriptyline, and/or at least one SSRI such as sertraline.

Preferably, the method comprises providing a topical dosage form comprising a composition having at least one FIASMA as an active ingredient, in a composition, and placing an effective amount of the composition onto a target area of skin to be treated, for a period of time required for delivery of the active ingredient to the site. In one preferred embodiment, the active ingredient or ingredients can be formulated as a viscous, controlled release gel, ointment, or cream for administration to the skin. A topical composition of the invention preferably comprises about 0.1% concentration (1 mg/ml of the composition) up to about 10% concentration (100 mg/ml of the composition) of each active ingredient provided; for example, 0.1%. 0.15%, 0.2%, 0.25%, 0.3%, 0.35%, 0.4%, 0.45%, 0.5%, 0.55%, 0.6%, 0.65%, 0.7%, 0.75%, 0.8%, 0.85%, 0.9%, 0.95%, 1.0% and 1.05%. 1.1%, 1.15%, 1.2%, 1.25%, 1.3%, 1.35%, 1.4%, 1.45%, 1.5%, 1.55%, 1.6%, and the like in 0.05% increments up to about 10.0%. A typical dose is about 0.5 ml to about 5 ml, preferably about 1 ml to about 2 ml, but is not so limited, and is bounded only by the area of skin needed to be treated, such that a dose of about 3 ml, 4 ml, 5 ml or greater can be applied. The topical formulation can be rubbed onto or into the target area of the skin prior to or following radiation therapy.

In another preferred embodiment, the FIASMA for topical administration is provided in an immediate-release topical formulation which allows targeted dosing and immediate delivery of the drug from the composition to the skin upon application or administration of the composition. Retention of the FIASMA component at the site of administration or application can be preferred. Accordingly, a composition of the invention may exclude a penetration enhancer to maintain the composition, and the active substance, on the surface of the skin for a longer period of time relative to a composition comprising a penetration enhancer which allows or facilitates penetration of the active substance into or below the skin where it can be absorbed systemically.

Alternatively, in certain instances, a penetration enhancer may be preferred and employed within the composition.

It would be understood that applying or administering the composition prior to radiation therapy can include retention of the composition at the location during the radiation therapy session. The method can be repeated for each radiation therapy session and can include administering or applying the composition at least one time per week, several times per week, daily, or multiple times daily, before, after, or between radiation therapy sessions. The method according to the subject invention can be continued for up to one month or up to one week after the radiation therapy sessions are discontinued. In an alternative embodiment, the method according to the subject invention can be continued for one week after radiation therapy sessions are discontinued.

According to one embodiment of the invention, the method of the invention comprises the step of:

topically administering an effective dose of one or more active ingredients, namely a drug which is a FIASMA, to the skin of a patient in need thereof, such as a patient who is suffering from CRI following exposure to environmental radiation or a patient that will undergo or is undergoing medical radiation therapy and is suffering from radiation dermatitis or radiation proctopathy.

In a method for preventing, reducing the incidence or recurrence of, inhibiting, treating, ameliorating, or reducing the severity of, or reversing oral mucositis or severe oral mucositis, the method can comprise the step of:
Providing a solid oral dosage form comprising a FIASMA as an active ingredient, and
administering to a patient in need thereof, such as a patient undergoing radiation treatment for head and neck cancer, a controlled release sublingual dosage form comprising a pharmaceutically effective amount of one or more active ingredients, wherein at least one active ingredient is a FIASMA.

In one embodiment, the solid oral dosage form is a modified release dosage form for sublingual administration comprising FIASMA as an active ingredient.

In another method for preventing, reducing the incidence or recurrence of, inhibiting, treating, ameliorating, or reducing the severity of, or reversing oral mucositis or severe oral mucositis, the method can comprise the step of:
Providing a solid oral dosage form comprising a FIASMA as an active ingredient, and
administering to a patient in need thereof, such as a patient undergoing radiation treatment for head and neck cancer, a controlled release buccal dosage form comprising a pharmaceutically effective amount of one or more active ingredients, wherein at least one active ingredient is a FIASMA.

In one embodiment, the solid oral dosage form is a modified release dosage form for buccal administration comprising FIASMA as an active ingredient.

In yet another method for preventing, reducing the incidence or recurrence of, inhibiting, treating, ameliorating, or reducing the severity of, or reversing oral mucositis or severe oral mucositis, the method can comprise the step of:
Providing a liquid oral dosage form or “rinse” comprising a FIASMA as an active ingredient, and
administering to a patient in need thereof, such as a patient undergoing radiation treatment for head and neck cancer, the oral rinse dosage form comprising a pharmaceutically effective amount of one or more active ingredients, wherein at least one active ingredient is a FIASMA

In a method for treating radiation proctopathy, the method of the invention comprises the step of:
anorectally administering an effective dose of one or more FIASMA drugs as an active ingredient, preferably a drug in the class selected from a TCA, an SSRI, or a combination thereof, to the rectal cavity or rectal tissue of a patient in need thereof, such as a patient suffering from radiation proctopathy or proctitis resulting from medical radiation treatment.

A preferred method for treating radiation proctopathy comprises anorectally administering an effective dose of a FIASMA as an active ingredient, such as the TCA, amitriptyline, the tricyclic antihistamine, loratadine, or the SSRI, sertraline. Compositions comprising a fixed dose combination two or more different FIASMAs, or one or more FIASMA with an active ingredient which is not a FIASMA can also be used. In one preferred example, the method is carried out using amitriptyline formulated as a composition and provided in dosage form which is capable of delivering an effective dose in situ to the rectal cavity of the patient. For example, amitriptyline can be formulated as a liquid and administered as an enema, or can be formulated with a thickening agent or viscosity-enhancing agent to provide a lotion, cream, ointment, or gel for topical delivery of amitriptyline to an anorectal area and rectal cavity of the patient.

Alternatively, the method can employ a semi-solid or solid dosage form, such as a suppository or a rectal plug delivery device for delivery of the active pharmaceutical ingredient to the anorectal cavity and in situ administration of the drug. Preferably, the dosage form provided with or incorporated into the rectal plug delivery device is a controlled release formulation, having excipients that can delay or retard the release of active drug incorporated into the dosage form or coated onto the outer surface of the dosage form.

Where the dosage form is a rectal plug delivery device, the device can be an insoluble plug forming a housing which has an internal chamber for containing therewithin a composition comprising the active pharmaceutical ingredient in accordance with the invention For example, the chamber of the rectal plug can be filled with a viscous composition comprising the FIASMA active ingredient or can contain a suppository dosage form comprising a FIASMA as the active ingredient.

An alternative embodiment of a rectal plug delivery device useful for carrying out the method of the invention comprises a porous compressible foam material infused or coated with a composition comprising the active pharmaceutical ingredient in accordance with the subject invention. The devices described for the method of the invention can contain an effective dose of the FIASMA provided in mounts of between about 0.1 mg to about 1000 mg or greater.

The method of the invention can further include the administration to a patient in need thereof of at least one active ingredient and an additional or second active ingredient which is not a FIASMA. Preferably, the additional active is formulated together with the at least one TCA, at least one SSRI, or a combination TCA and SSRI in a fixed-dose combination drug product.

One preferred embodiment of composition having a first and second active comprising a TCA or an SSRI and an anti-inflammatory, anesthetic agent, or an antioxidant. The anti-inflammatory can be a steroid or a non-steroidal anti-inflammatory (NSAID). Alternatively, the second active in the composition is not an anti-inflammatory agent, wherein the composition excludes a steroid compound, or the composition excludes a non-steroidal anti-inflammatory drug (NSAID). The anesthetic agent can be a local anesthetic commonly used for topical administration, such as lidocaine. The antioxidant can be, for example, Vitamin A, Vitamin C, Vitamin D, and Vitamin E.

The subject invention includes a composition for treating radiation proctopathy comprising providing an effective amount of a FIASMA formulated as a controlled release formulation for in situ delivery of the active ingredient to the rectal cavity. In one preferred embodiment, the composition can be formulated as a controlled release suppository containing at least one TCA or at least one SSRI, or at least one TCA and one SSRI, as the active ingredient or ingredients. The composition can include an additional drug which is not a FIASMA as described herein.

The active pharmaceutical ingredient contained in a suppository or rectal delivery device can be administered prior to, during, or following pelvic radiation therapy. A preferred administration includes inserting the suppository or rectal delivery device into the rectal cavity and allowing the suppository or rectal delivery device to remain within the rectal cavity until delivery of the entire dose is completed. A preferred suppository in accordance with the invention can slowly dissolve and remain for a period of up to about 24 hours, i.e., daily use. A rectal plug delivery device can be retained within the rectal cavity for a period of up to about 48 hours but is preferred to include a drug formulation which delivers the dose of the active ingredient or ingredients within about 24 hours. This provides for daily use following a bowel movement. Alternatively, the rectal plug can be temporarily removed for bowel movements and re-inserted.

The suppository containing active pharmaceutical ingredient is placed to reside within the rectal cavity for the time period required for dissolution. Since the suppositories are fully dissolvable, release of active ingredient from the suppository will be achieved after residence of the suppository in the rectum. The released active ingredient will occur in high concentrations at the site of delivery, thus enhancing the effectiveness of this therapy for anorectal disorders.

It would be understood that applying or administering the composition prior to radiation therapy can include retention of the composition at the location during the radiation therapy session. The method can be repeated for each radiation therapy session and can include administering or applying the composition at least one time per week, several times per week, daily, or multiple times daily, before, after, or between radiation therapy sessions. The method according to the subject invention can be continued for up to one month or up to one week after the radiation therapy sessions are discontinued. In an alternative embodiment, the method according to the subject invention can be continued for one week after radiation therapy sessions are discontinued.

According to an alternative embodiment, the suppository may comprise or consist of other drugs or supplements, such as antioxidants, including vitamin E and vitamin C and natural antioxidants such as fish oils, green tea, cranberry, and the like. These may be used as distinct suppository preparations or as additional components of the suppositories of the invention. These uses and in any of the embodiments of the invention, a suppository form of these agents is used as a clinical treatment for chronic diseases of the anus and rectum.

Any form of the active ingredients contemplated by the invention that is placed in a suppository form for the treatment of anorectal diseases is within the confines of the invention. Additionally, the incorporation of active pharmaceutical ingredients described herein in combination with other active or inactive ingredients into suppositories to treat anorectal diseases is embodied within this invention. Additionally, the incorporation of any FIASMA within the suppositories as a means of treating anorectal disorders is embodied in the invention. Finally, other agents, such as anti-inflammatoires, anesthetics, herbals, or other vitamins may be included in the suppositories to enhance the efficacy of the active ingredient or ingredients. Substances utilized to produce the suppositories include any fatty (or oleaginous) bases and/or water soluble (or miscible) bases.

In particularly preferred embodiments, the medication contained in the suppository is amitriptyline. In the preferred embodiment, the suppository is composed of fatty (or oleaginous) bases and/or water soluble (or miscible) bases. However, other bases may be employed in the invention to allow for the passage of the medication into the rectum. More generally, any form of suppository base may be used to construct the devise. In addition, a variety of TCAs may be incorporated into the suppository to allow direct application of these substances to the rectum and anus. Other aforementioned substances may also be included in the suppositories to enhance their efficacy in treating anorectal disorders. The contents of the suppository may also consist of a variety of TCAs, either alone or in combination with amitriptyline, depending on the goal of treatment.

Variable doses of amitriptyline may be utilized, depending on the condition being treated. For example, the rectal dose of amitriptyline used for the treatment of radiation proctopathy is 25-100 mg a day. Both lower and higher doses than these would initially be employed in the construction of the device. The optimal dosage to treat these conditions will be determined based on clinical studies.

However, following appropriate clinical evaluation of this treatment, either larger or smaller doses of amitriptyline may ultimately be used for treating radiation proctopathy as well as other anorectal disorders. Dosing for amitriptyline and other TCAs in suppositories are anticipated to be lower than oral doses for the treatment of anorectal diseases because these agents will be directly applied to the affected areas. However, because the delivery by suppository or rectal device does not need to be ingested and the dosage forms can be larger in size, higher doses than typically employed in oral dosage forms may also be utilized if determined safe and effective based on further clinical experience or studies.

Preferably, the method for treating CRI is carried out using a FIASMA, e.g., a TCA such as amitriptyline, a tricyclic antihistamine, such as loratadine, or an SSRI, such as sertraline, wherein the TCA, tricyclic antihistamine, or SSRI or a combination of at least two of the active ingredients are prepared as a composition and provided in a dosage form which is capable of delivering an effective dose in situ to the skin of the patient. For example, amitriptyline can be formulated as a liquid and administered in liquid form to the target area of the skin, or can be formulated with a thickening agent or viscosity-enhancing agent to provide a lotion, cream, ointment or gel for topical delivery of amitriptyline to target area of the skin of a patient in need thereof, such as a patient who will undertake radiation therapy or who is suffering from radiation dermatitis.

Alternatively, the method can employ administration of a solid oral dosage form.

The method of the invention can further include the administration to a patient in need thereof of at least one FIASMA and an additional active ingredient which is not a FIASMA. Preferably, the additional active is formulated together with the at least one FIASMA in a fixed-dose combination drug product for topical administration to the skin.

One preferred embodiment of a composition of the invention having a first and second active comprises an SSRI as a first active ingredient and a TCA, tricyclic antihistamine, or an antioxidant as a second active ingredient. The antioxidant can be, for example, Vitamin A, Vitamin C, Vitamin D, and Vitamin E. A further embodiment of a composition of the invention can comprise a TCA, an SSRI, and an antioxidant. Examples of the active ingredients are amitriptyline as the TCA, loratadine as the tricyclic antihistamine, sertraline, as the SSRI, and Vitamin D as the antioxidant.

Variable doses of the one or more active ingredient described herein may be utilized, depending on the condition being treated. For example, the topical dose of amitriptyline used for the prevention or treatment of radiation dermatitis is 1 mg-100 mg a day.

The optimal dosage and area to be treated to prevent or treat these conditions will be determined based on clinical studies. However, following appropriate clinical evaluation of this treatment, either larger or smaller doses of amitriptyline or sertraline may ultimately be used for preventing or treating radiation dermatitis as well as other dermatological inflammatory disorders.

### EXAMPLES

### Example 1—Use of a Composition Comprising a TCA

A patient undergoing or scheduled to undergo radiation therapy for treatment of a 25 mm breast tumor will be provided a composition which is an ointment comprising 1%-2% amitriptyline in a pharmaceutically acceptable base. The area of the skin over the tumor, which is or will be exposed to the radiation during the radiation therapy procedure will be identified and can be marked.

The healthcare worker or patient will administer the composition by applying approximately 1 ml of the composition to the area of the skin exposed or expected to be affected by the radiation at least one time per day. Administration of the composition will be repeated at least daily, or up to five times daily during the radiation treatment regimen for a period of at least one week. For example, a patient undergoing a radiation therapy regimen five times per week (Monday through Friday), may apply cream to the treated area 90 minutes before radiation therapy each day and then at bedtime, for seven days. So before and after the radiation treatment Monday to Friday and then twice a day on the weekends when they are not getting radiation. This is repeated for 5 or 6 weeks.

Expected result: radiation dermatitis is prevented or ameliorated or reversed by the administration of the composition.

### Example 2—Determining Efficacy of Single Active v a Plurality of Actives

Five patient groups undergoing or scheduled to undergo radiation therapy for treatment of a 25 mm breast tumor will be provided a composition which is an ointment comprising either:
1% amitriptyline in a pharmaceutically acceptable base;
1% sertraline in a pharmaceutically acceptable base;
1% amitriptyline and 1% sertraline in a pharmaceutically acceptable base;
0.5% amitriptyline and 0.5% sertraline in a pharmaceutically acceptable base; and
Pharmaceutically acceptable base, alone (placebo).

The area of the skin over the tumor, which is or will be exposed to the radiation during the radiation therapy procedure will be identified and can be marked. The healthcare worker or patient will administer the composition by applying approximately 1 ml of the composition to the area of the skin exposed or to be exposed to the radiation at least one time per day before radiation treatment. Administration of the composition will be repeated at least daily, or up to five times daily following the radiation treatment for a period of one week.

Expected results: The efficacy for each composition comprising an active ingredient will be determined by scoring the level of radiation dermatitis present in each group of patients. A determination of whether the efficacy of using amitriptyline and sertraline in combination exhibits an additive or synergistic effect can be determined by comparing whether the radiation dermatitis score for the composition comprising 1% amitriptyline and 1% sertraline is less than, equal to, or greater than the effect of the 1% amitriptyline composition, alone, and the 1% sertraline composition, alone. The radiation dermatitis score for the fixed dose combination composition comprising 0.5% amitriptyline and 0.5% sertraline in a pharmaceutically acceptable base can provide information on efficacy of lower doses of each active, in combination, compared to higher doses of each active, alone, in a composition.

### Example 3—Treatment of Radiation Dermatitis in Radiated Mice

Purpose: Determining effects of topical reformulation of sertraline, as a protector for radiation-induced skin injury.

Materials and Methods: Radiation-induced dermatitis experiments were conducted in 10-week-old female C57BL/6J mice (The Jackson Laboratory). An approximately 3×3 cm area on the dorsal skin of mice was shaved prior to drug treatment. Mice were randomly assigned to receive topical treatment of 4% sertraline or vehicle (n=5 mice per cohort) and all investigators were blinded as to topical treatment.

The study compared active drug-containing composition to a placebo (vehicle). Both the active drug-containing composition and placebo were prepared by a third party and provided to us for use in the study. The preparation of compositions is summarized as follows:
4% Sertraline Cream—4% (w/w) Sertraline (as a hydrochloride salt) in an oil-in-water vanishing cream base comprising hexylene glycol, purified water, isopropyl palmitate, caprylic/capric triglyceride, propylene glycol, ceteareth 20, cetearyl alcohol, glyceryl stearate, PEG 100 stearate, dimethicone, octyldodecanol, lecithin, ethylhexylglycerin, and phenoxyethanol. Placebo Cream—An oil-in-water vanishing cream base comprising purified water, isopropyl palmitate, caprylic/capric triglyceride, propylene glycol, ceteareth 20, cetearyl alcohol, glyceryl stearate, PEG 100 stearate, dimethicone, octyldodecanol, lecithin, ethylhexylglycerin, and phenoxyethanol. Both products were packaged in white polypropylene jars with white polypropylene closures with polyvinyl chloride disc liners, labeled for external use only, storage at controlled room temperature (20° C. to 25° C.) and with instruction to not freeze.

The formulation is further described in Composition Table A, below:

Composition Table A.
Rythera Topical Cream Formulations
Composition-4% Sertraline

| Component | Lot | % w/w | 220 g |
|---|---|---|---|
| Sertraline HCl | 6653I-NJ | 4.48 | 9.85 |
| Hexylene Glycol | NG1ST364 | 5.00 | 11.00 |
| PENCream | A60093 09 2023 | 90.52 | 199.15 |
| | Total | 100.00 | 220.00 |
| Sertraline HCl MW | 342.69 | | |
| Sertraline FB MW | 306.23 | | |

Instructions
1) Add Sertraline HCl to a glass mortar.
2) Add hexylene glycol.
3) Triturate using the glass pestle.
4) Add PEN Cream base in aliquots to the glass mortar and mix.
5) Package in 4 oz white PP double wall radius jar with white PP dome cap and white PVC disc liner 70/400
SKS Part No. 0661-21

As noted in the Composition Table A, the 4% sertraline composition is prepared as the free base. The amount of sertraline HCl added to the formulation is adjusted to compensate for the HCl salt.

PENCream is a commercially available oil-in-water vanishing base purchased from Humco (https://www.humco.com/pharmaceuticals/pencream/). The NDC code of the cream is 0395-6010-56.

Mice received topical treatment once per day for 12 days starting at 2 days before irradiation. Dermatitis was induced by delivering a single dose of 30 Gy X-rays to a 1×1 cm area of the shaved skin using an image-guided small animal irradiation X-Rad 225 Cx.

The development of radiation-induced dermatitis was examined before irradiation and every week for 4 weeks after irradiation by a single observer who was blinded to the treatment groups. Skin reaction to radiation was assessed according to a semi-quantitative score system established previously for preclinical studies—scoring ranges from 1.0 to 5.5 (0.5 increments) based on erythema, desquamation (dry and moist), necrosis and loss of dermis (Table 1).

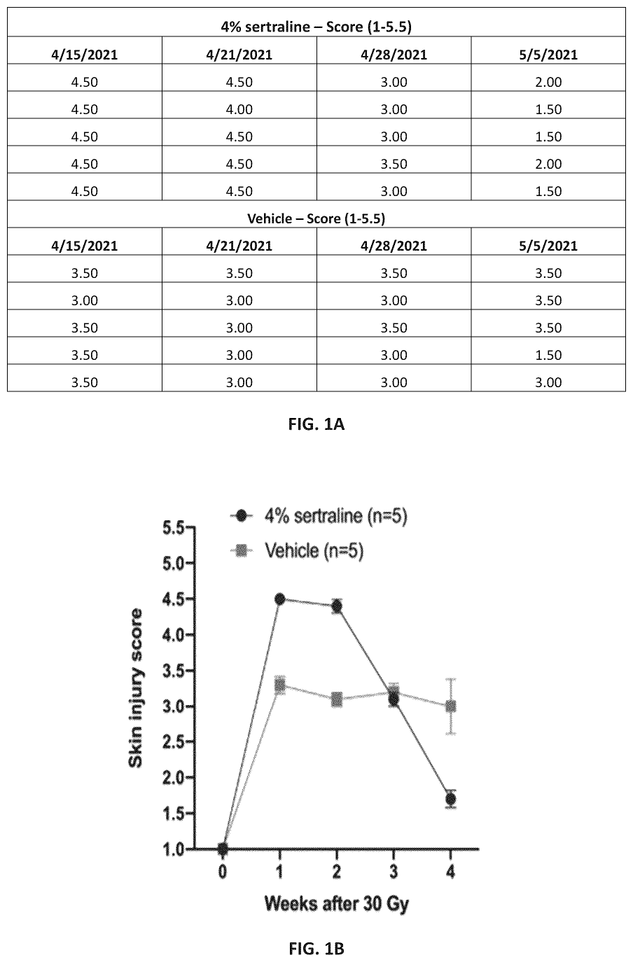

Results: During the first two weeks after 30 Gy irradiation, all of the mice that received treatment with 4% sertraline cream had a skin injury score of 4.5, while the mice that received vehicle treatment had less severe scores of 3.0-3.5 (FIG. 1, panels A and B). Scratching behaviors and sequelae were noted in the 4% sertraline cohort.

On day 21 after irradiation, both treatment cohorts had skin injury scores of 3.0-3.5. At the end of the experiment on day 28 after irradiation, mice that received treatment of 4% sertraline had less severe skin injury scores ranging from 1.5-2.0 with an average score of 1.7.

In contrast, vehicle treated mice maintained higher skin injury scores ranging from 1.5-3.5 with an average score of 3.0 (FIG. 1, panels A and B).

Discussion: Our preliminary results show that skin injury of mice that received 4% sertraline was substantially improved 4 weeks after 30 Gy, whereas skin injury of vehicle treated mice remained severe. The causes of higher injury scores for the mice treated with 4% sertraline during the first two weeks after irradiation are under investigation, but literature search has confirmed that in mice, topical sertraline trigger a significant itch-evoked scratching in mice as shown in previous published animal studies (Neuron 87, 124-138, Jul. 1, 2015). Repeat of this experiment with removal of cream after 30 minutes substantially reduced the scratching behavior and initial skin injury score while still showing similar benefit after 2 weeks as the data shown above. Therefore, it is believed that the early toxicity noted at week 1 was related to self-injury due to sertraline-induced scratching.

Conclusion: The results surprisingly suggest that a 4% FIASMA composition, e.g., 4% sertraline topical composition, is efficacious against radiation-induced skin injury.

Compositions comprising lower concentration of active drug, e.g., less than 1% sertraline, preferably about 0.5%, wherein the compositions are formulated with one or more penetration enhancer, solubilizer, emollient, or the like, can deliver substantially the same amount of drug to the target site as the composition described in Composition Table A, above. These formulations comprising 1% or less active drug may be advantageous regarding dosage to a patient, and therefore preferred.

A topical gel composition for delivering a therapeutically effective amount to a target site of the skin of a patient can include, for example, 0.5-1.0% active agent in a mixture with one or more preservative, one or more humectant/solvent, one or more rheology modifier/thickener, one or more anti-tack agent/glidant, and the like. An example of a formulation of a topical gel is provided in GEL COMPOSITION TABLE B, below.

GEL COMPOSITION TABLE B

| Ingredient | % (w/w) |
|---|---|
| KOLLIPHOR EL | 1 |
| PHENOXY-2 ETHANOL | 0.5 |
| ALCOOL BENZYLIQUE EMPROVE EXPERT PH | 1 |
| GLYCERINE | 5 |
| PROPANEDIOL-1,2 | 5 |
| HEXYLENE GLYCOL PHARMA USP/NF | 5 |
| CARBOPOL ETD 2020 NF | 0.2 |
| PEMULEN TR1 NF | 0.4 |
| PURIFIED WATER | QS 100% |
| MICRONIZED SERTRALINE HYDROCHLORIDE | 0.5595 |
| XIAMETER PMX-200 SILICON FLUID 5 CST | 20 |
| SODIUM HYDROXIDE 10% (w/w) SOLUTION | QS pH 5 ± 0.5 |

A topical cream formulation for delivering a therapeutically effective amount to a target site of the skin of a patient can include, for example, 0.5-1.0% active agent in a mixture with one or more preservative, one or more humectant/solvent, one or more rheology modifier/thickener, one or more anti-tack agent/glidant, and the like.

An example of a formulation of a topical gel is provided in CREAM COMPOSITION TABLE C, below.

CREAM COMPOSITON TABLE C

| Ingredient | % (w/w) |
|---|---|
| PHENOXY-2 ETHANOL | 0.5 |
| ALCOOL BENZYLIQUE EMPROVE EXPERT PH | 1 |
| GLYCERINE | 5 |
| PROPANEDIOL-1,2 | 10 |
| CARBOPOL ETD 2020 NF | 0.2 |
| XANTHAN GUM FF | 0.3 |
| PURIFIED WATER | QS 100% |
| MICRONIZED SERTRALINE HYDROCHLORIDE | 0.5595 |
| SP BRIJ S721 MBAL-PA-(SG) | 1 |
| SP BRIJ S2 MBAL-SO | 4 |
| KOLLIWAX HCO | 2 |
| KOLLIWAX SA | 2 |
| MIGLYOL 812N | 10 |
| Q7-9120 SILICONE FLUID 350 CST | 1 |
| SODIUM HYDROXIDE 10% (w/w) SOLUTION | QS pH 5 ± 0.5 |

### Example 4-—Comparison of Pre-Irradiation and Post-Irradiation Treatments

The experiment presented in Example 3 was repeated in mice to compare 4% sertraline administered pre-irradiation treatment and post irradiation treatment. 4% amitriptyline was also tested.

The same scoring chart shown in Example 3 was used to evaluate the irradiated sites on the mice.

Methods: Radiation-induced dermatitis experiments were conducted using 10-week-old female C57BL/6J mice (The Jackson Laboratory). An approximately 3×3 cm area on the dorsal skin of mice was shaved prior to drug treatment. A single dose of 30 Gy X-rays was delivered to a 1×1 cm area of the skin using an image-guided small animal irradiator X-Rad 225 Cx to induce dermatitis. Mice were randomized by cage to receive topical treatment of 4% sertraline cream once a day for 3 days before irradiation, 4% sertraline cream once a day for 2 days starting at 24 hours after irradiation or placebo once a day for 5 days (before and after irradiation).

The development of radiation-induced dermatitis was examined every week for 6 weeks after irradiation by two observers who were blinded to the treatment cohorts (S.H. and S.S). Skin reaction to radiation was assessed according to a semi-quantitative score system established previously for preclinical studies—scoring ranges from 1.0 to 5.5 (0.5 increments) based on erythema, desquamation (dry and moist), necrosis and loss of dermis.

The score results at 1 week, 2 weeks, 3 weeks, and 4 weeks post irradiation treatment (IR) are shown in Table 2, below:

TABLE 2
Semiquantitative Skin Damage Scores

| | | | | |
|---|---|---|---|---|
| | 1 week | 2 weeks | 3 weeks | 4 weeks |
| Mouse No. | post IR | post IR | post IR | post IR |

A. 4% sertraline cream applied 48 hr., 24 hr.,
and 30 min. prior to irradiation treatment

| | | | | |
|---|---|---|---|---|
| 6632 | 4.00 | 4.00 | 3.50 | 2.00 |
| 6633 | 4.00 | 4.00 | 4.00 | 2.00 |
| 6634 | 4.00 | 4.00 | 4.50 | 4.50 |
| 6635 | 4.00 | 4.00 | 3.00 | 2.50 |
| 6636 | 4.00 | 4.00 | 4.00 | 2.50 |

B. Vehicle-only cream applied 48 hr., 24 hr., and 30 min.
prior and 24 hr. and 48 hr. post irradiation treatment

| | | | | |
|---|---|---|---|---|
| 6637 | 4.00 | 4.00 | 4.00 | 3.00 |
| 6638 | 3.50 | 3.50 | 3.50 | 3.00 |
| 6639 | 4.00 | 4.00 | 3.50 | 3.50 |
| 6640 | 4.00 | 4.00 | 4.00 | 4.00 |
| 6641 | 3.00 | 3.00 | 4.00 | 4.00 |

C. 4% sertraline cream applied 24 hr. and
48 hr. post irradiation treatment

| | | | | |
|---|---|---|---|---|
| 6642 | 2.50 | 4.00 | 2.50 | 2.00 |
| 6643 | 3.00 | 3.00 | 3.00 | 1.50 |
| 6644 | 2.50 | 3.50 | 3.00 | 3.00 |
| 6645 | 3.00 | 1.50 | 1.50 | 1.50 |
| 6646 | 3.00 | 1.50 | 1.50 | 1.50 |

RESULTS. At 4 weeks post-irradiation treatment, 9 of 10 sertraline-treated mice showed lower scores (better improvement) than vehicle-only treated mice. Generally, the 4% sertraline cream performed better when administered post-irradiation treatment than pre-irradiation treatment; however, pre-irradiation treatment exhibited some preventive effects. 4% sertraline cream administered 24 hr. and 48 hr. post-irradiation treatment also showed an earlier response, showing lower scores within one week of irradiation treatment, and continuing to show improved scores through the fourth week after irradiation treatment.

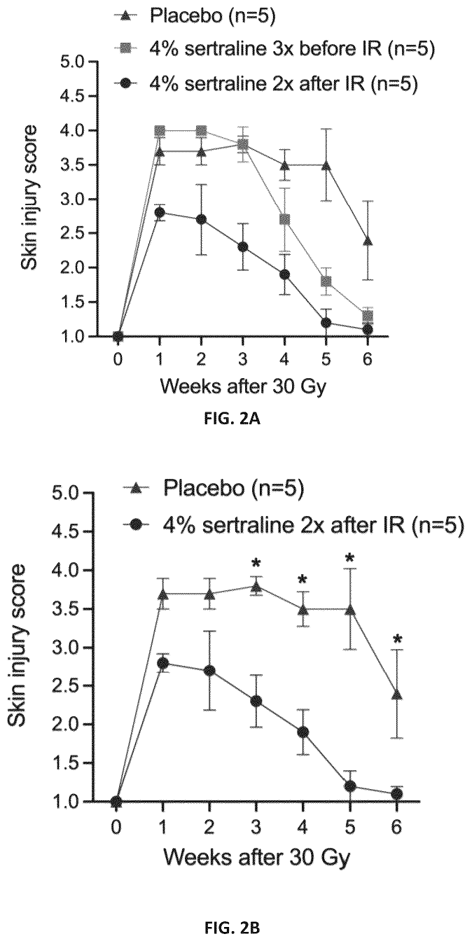

These results are graphically represented in FIG. 2, panels A-D. Overall, mice that received 4% sertraline 2 times after irradiation showed accelerated recovery from acute radiation dermatitis starting at the 1st week after irradiation (FIG. 2, panels A, B, and D). The skin injury scores for the 2× after group were significantly lower than the placebo group at week 3 to 6 after 30 Gy (FIG. 2, panel B). On the other hand, the skin injury scores for the 3× before group were significantly lower than the placebo group only at week 5 after 30 Gy (FIG. 2, panel C). Remarkably, mice that received 4% sertraline 3 times before irradiation showed significantly slower recovery from acute radiation dermatitis compared with mice that received 4% sertraline 2 times after irradiation (FIG. 2, panel D).

Notably, administration of 4% amitriptyline cream and 4% fluoxetine cream had deleterious effects on the mice and this arm of the experiment was stopped. This suggests that a 4% cream formulation comprising a tricyclic antidepressant (TCA) is an excessive dose and that lower doses are required to determine their efficacy.

### Example 5—Oral Solution or Rinse Comprising an SSRI for Treating Oral Mucositis Associated With Head and Neck Radiation Therapy

Purpose: The goal of this planned study is to evaluate the effectiveness of an oral solution or rinse comprising an SSRI (sertraline) for treating oral mucositis and xerostomia associated with head and neck radiation therapy.

### A. Optimal Radiation Dose for Inducing Oral Mucositis in Mouse Model

Pilot experiments will be performed to determine the optimal dose of irradiation to study radiation-induced oral mucositis and xerostomia in 8-week-old female C57BL/6J mice. The head-and-neck region of mice will be treated with a single dose of 0, 16, 18, 20 and 22 Gy X-rays using the Small Animal Radiation Research Platform (SARRP) (n=10 per radiation dose).

Two measurements will be performed to assess the duration and severity of oral mucositis:
(a) The body weight of mice will be measured every day from day 7 to day 16 after irradiation and three times a week thereafter for up to 21 days after irradiation and
(b) Pathomorphological changes in the tongue will be assessed using an otoscope on day 9, 12 and 16 after irradiation.

Pain control medicine and supportive care may be provided if needed.

To assess the severity of xerostomia, the production of saliva per mouse will be measured 30 days and 60 days after irradiation according to method previously described. See, Ashcraft et al. (2015) “Novel Manganese-Porphyrin Superoxide Dismutase-Mimetic Widens the Therapeutic Margin in a Preclinical Head and Neck Cancer Model.” International Journal of Radiation Oncology, Biology, Physics 93 (4): 892-900; Saiki et al. (2018) “Aldehyde Dehydrogenase 3A1 Activation Prevents Radiation-Induced Xerostomia by Protecting Salivary Stem Cells from Toxic Aldehydes.” Proceedings of the National Academy of Sciences of the United States of America 115 (24): 6279-84.

### B. Determining the Effect of Sertraline Oral Solution on the Development of Radiation-Induced Oral Mucositis and Xerostomia

Using the optimal radiation dose determined in A., above, 8-week-old female C57BL/6J mice will be randomly assigned to receive
head and neck radiation therapy, plus vehicle only (control),
head and neck radiation therapy+2% sertraline in vehicle,
head and neck radiation therapy+4% sertraline in vehicle and
head and neck radiation therapy+6% sertraline in vehicle
   (n=20 mice/treatment cohort).

Mice will be treated with sertraline oral solution once a day for 5 consecutive days (2 days before irradiation, the same day immediately before irradiation, and 2 days after irradiation).

Two measurements will be performed to assess the duration and severity of oral mucositis:
(a) The body weight of mice will be measured every day from day 7 to day 16 after irradiation and three times a week thereafter for up to 21 days after irradiation.
(b) Pathomorphological changes in the tongue will be assessed using an otoscope on day 9, 12 and 16 after irradiation. Pain control medicine and supportive care may be provided if needed. To assess the severity of xerostomia, the production of saliva per mouse will be measured 30 days and 60 days after irradiation (Ashcraft et al. 2015; Saiki et al. 2018). Fibrosis of the salivary glands will be assessed 60 days after irradiation by histology (Ashcraft et al. 2015).

It is hypothesized that oral treatment with sertraline will decrease the severity and/or duration of radiation-induced oral mucositis and xerostomia by reducing acute inflammation. It is expected that the results will be dose-dependent and that sertraline oral solution will be effective in treating oral mucositis and/or xerostomia.

### Example 6—Treatment of Radiation Dermatitis in Patients Undergoing and Recovering From Radiation Therapy

Purpose: Determining effects of RAD-100 in the prevention or reduction of radiation-induced dermatitis in post-surgical breast cancer patients undergoing a standard course of radiation therapy.

Materials and Methods: A randomized, double-blind study was conducted using subjects aged 18 years or older who recently underwent breast cancer surgery—either breast conservation (lumpectomy) or mastectomy—and who were scheduled to undergo a standard course of radiation therapy of 1.8 to 2.0 GY per session for between 25-30 sessions over a five to six-week period administered as 5 consecutive daily treatments per week.

Subjects were excluded from the study if they met any one of the following exclusion criteria:
Ulcerated breast tissue or open breast wounds at the beginning of radiation therapy.
Skin lesions in the area scheduled for radiation before initiation of radiation.
Known allergies, hypersensitivity or reaction to any of the components of the cream or to sertraline.
Any neurological or psychiatric conditions that in the opinion of the treating physician deem the subject incapable of participation in the study.
Any other significant comorbidity (e.g., uncontrolled diabetes, renal failure, hepatic dysfunction, cardiovascular disease, etc.) which in the opinion of the Investigator could affect the outcome of the study.
BMI>35.0
Subjects involved in an investigational drug trial within the past thirty (30) days.
Subjects taking a monoamine oxidase inhibitor.
Subjects undergoing concurrent treatment with a chemotherapeutic drug.
Pregnant or lactating.

Subjects who met all the inclusion criteria and none of the exclusion criteria were enrolled in the study and were required to read, understand and sign an informed consent, complete a medical questionnaire, undergo a complete physical examination, provide a fasting blood sample for chemistry and hematology, and a urine sample for urinalysis.

The study consisted of a cohort of 14 subjects randomized 2:1 to receive RAD-100 (sertraline topical cream 4.00% w/w) or vehicle cream twice a day (BID). Subjects were be asked to apply the cream twice daily, once in the morning, and once in the evening and to continue the regimen every day during the radiation treatment and for 14 days following the last radiation treatment.

Subjects were dispensed the cream on the Friday prior to the first week of radiation therapy, with instructions to start application of the cream on the Sunday evening prior to their first radiation treatment. One day prior to starting a standard course of radiation therapy, all subjects were be asked to start the application of cream to a defined area where the radiation was to be administered. This included the whole breast for the breast conservation subjects and a defined area in the mastectomy subjects.

Subjects were asked to apply the cream twice daily, once in the morning, and once in the evening and to continue this regimen every day during radiation treatment and for 14 days following the last radiation treatment. All subjects received blinded coded medication according to a predetermined randomization code in the ratio of two active to one vehicle control that was unknown to both the subject and the investigator/evaluator. All subjects were provided with a diary to record the times at which they applied the cream and to record any concomitant medication and adverse events. Photographs of the application site were taken twice weekly on Tuesday and Friday prior to radiation treatment and on the 7th and 14th day after radiation treatment.

Following the initial one-day run-in all subjects reported daily, five days a week to the radiation department to receive their radiation dosage. Immediately prior to the radiation therapy treatment every Tuesday and Friday, the Principal Investigator (PI) or a designated assistant of the PI evaluated the skin at the site of the radiation and record a score using the revised Radiation Therapy Oncology Group (RTOG) scoring system. Subjects were questioned weekly concerning the radiation site and were asked to report pain level, as well as any adverse or unusual events. Subjects were reminded to continue application of the cream during the two days a week they did not receive radiation and to note any changes in the radiation treatment site in a diary. At the completion of the radiation phase of the study all subjects were asked to continue the treatment regimen daily for the next 14 days. Subjects returned on the seventh and fourteenth day for evaluation. At the end of this 14-day post-radiation period subjects were discharged from the study with a request to voluntarily report any subsequent changes at the radiation site.

Results: Dermatitis did not develop earlier than Day 26 except in a few subjects. The results showed P-values favorable to the treated group throughout the treatment period and into the post-radiation period. While radiation induced dermatitis as recorded in the RTOG score worsened in the placebo group during the 7-day post radiation period, the mean score stabilized in the treated group. Table 3 shows the mean RTOG score data from Day 26 onward.

TABLE 3
Mean RTOG Score

| | RTOG (Treated | | RTOG Placebo | | |
|---|---|---|---|---|---|
| Day | Group) | N | Group | N | P |
| 26 | 0.7 | 10 | 1.0 | 4 | 0.28 |
| 29 | 1.0 | 10 | 1.0 | 4 | 1.00 |
| 33 | 1.0 | 10 | 1.5 | 4 | 0.13 |
| 36 | 1.2 | 10 | 1.5 | 4 | 0.46 |
| 7-days post | 1.2 | 10 | 2.0 | 4 | 0.27 |
| 14-days post | 0.9 | 10 | 1.0 | 4 | 0.81 |

The distribution of RTOG scores within each group at different times was also evaluated. The dermatitis worsened earlier in the placebo group compared to the treated group and all P-values were in favor of the treated group. In the post-radiation phase, the treated group appeared to heal faster. Table 4 shows the distribution of the RTOG score by category.

TABLE 4
Categorical RTOG scores (% of subjects with a particular score

| | RTOG | % Treated | | RTOG | % Placebo | | |
|---|---|---|---|---|---|---|---|
| Day | Score | Group | N | Score | Group | N | P |
| 26 | 0 | 30 | 10 | 0 | 0 | 4 | 0.22 |
| | 1 | 70 | | 1 | 100 | | |
| | 2 | 0 | | 2 | 0 | | |
| 29 | 0 | 10 | 10 | 0 | 0 | 4 | 0.63 |
| | 1 | 80 | | 1 | 100 | | |
| | 2 | 10 | | 2 | 0 | | |
| 33 | 0 | 10 | 10 | 0 | 0 | 4 | 0.24 |
| | 1 | 80 | | 1 | 50 | | |
| | 2 | 10 | | 2 | 50 | | |
| 36 | 0 | 10 | 10 | 0 | 0 | 4 | 0.68 |
| | 1 | 60 | | 1 | 50 | | |
| | 2 | 30 | | 2 | 50 | | |
| 7-days post | 0 | 33 | 9 | 0 | 0 | 4 | 0.49 |
| | 1 | 22.6 | | 1 | 25 | | |
| | 2 | 44.4 | | 2 | 75 | | |
| 14-days | 0 | 25 | 8 | 0 | 25 | 3 | 0.63 |
| post | 1 | 63 | | 1 | 50 | | |
| | 2 | 12 | | 2 | 25 | | |

While RTOG does include scores higher than 2, radiation treatments are designed to not exceed score of 2. Although data is presented as the percentage of patients in each group who scored 2 or higher, in reality, no score exceeded a score of 2.

The data in Table 5 (below) shows the categorized data for this study. During the radiation treatment phase, the placebo group had a higher percentage of 2.0 scores at day 33 and 36 with close statistical significance at Day 33. The placebo group also showed a poorer recover.

TABLE 5
Categorized RTOG data (% of subjects with a score of 2 or >2)

| | % Treated | | % Placebo | | |
|---|---|---|---|---|---|
| Day | Group | N | Group | N | P |
| 26 | 0 | 10 | 0 | 4 | — |
| 29 | 10 | 10 | 0 | 4 | 0.52 |
| 33 | 10 | 10 | 50 | 4 | 0.10 |
| 36 | 30 | 10 | 50 | 4 | 0.48 |
| 7-day post | 44 | 9 | 75 | 4 | 0.31 |
| 14-day post | 12 | 8 | 33 | 3 | 0.43 |

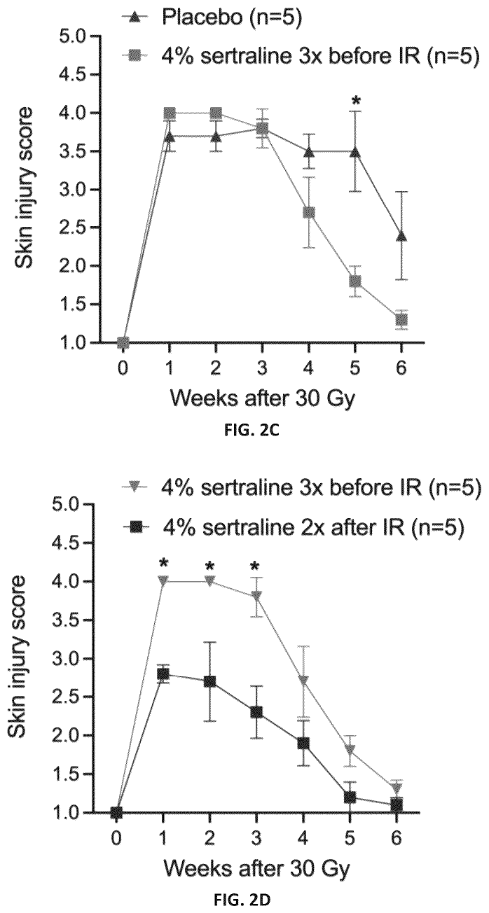

The results of Table 5 are graphically represented in FIG. 3.

Conclusion: The results of this study have shown that treatment using RAD100 improves the course of dermatitis during both the radiation treatment phase and the recovery phase, achieving some near significant P-values. While larger number of subjects will inevitably improve the statistical data of this study, current values predict that larger studies will achieve high statistical significance. Overall, the delayed occurrence of dermatitis as well as the treated group's rapid rate of healing compliments the data which were generated with mice in Examples 3 and 4. Mice data, like human data, shows an improved rate of healing in the RAD100 treated group.

### Example 7—Treatment for Tissue Toxicity Associated With Head and Neck Radiation Therapy

Purposes: Determining the impact of sertraline on the development of radiation-induced oral mucositis in mice by examining the effect of sertraline oral solution on the development of radiation-induced oral mucositis in mice.

Experimental procedure: Ten weeks old female C57BL/6 mice were randomly assigned by cage to receive 16 Gy oral irradiation+placebo or 16 Gy oral irradiation+1% sertraline solution. The placebo or sertraline solution was applied to the oral cavity of mice once a day for 5 consecutive days (2 days before irradiation, the same day 30 minutes to 1 hour before irradiation and 2 days after irradiation). The development of radiation-induced severe oral mucositis was followed for 21 days after irradiation.

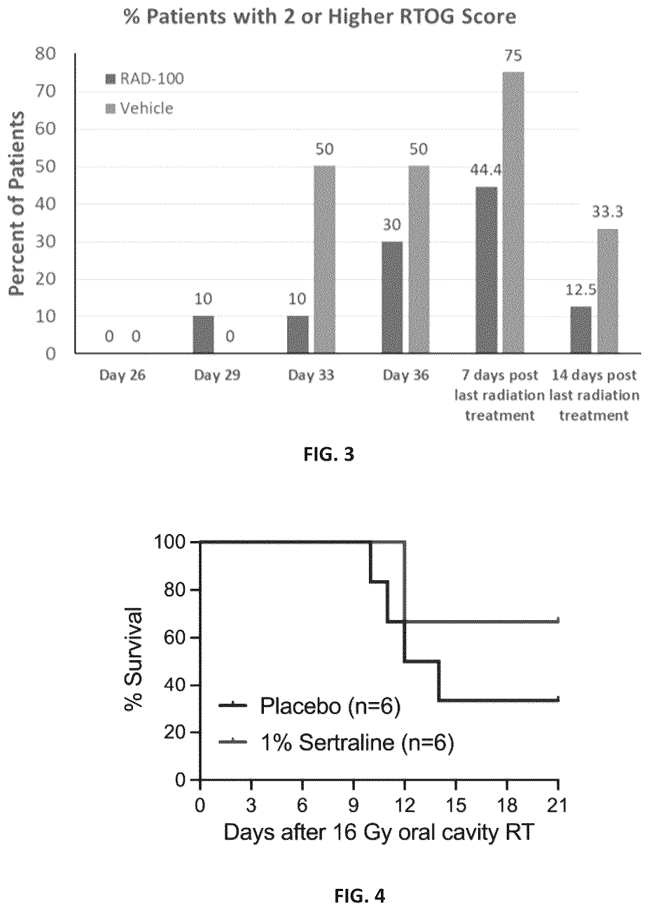

Results: The results showed that in the placebo group 16 Gy oral irradiation caused 66% (4 out of 6) of mice died due to acute radiation toxicity of the oral mucosa, whereas only 33% (2 out of 6) of mice died in the 1% sertraline group (FIG. 4). These findings support the development of oral sertraline treatment as a novel approach to ameliorate radiation-induced severe oral mucositis.

The foregoing description is illustrative only and is not intended to limit the scope of the invention to the precise terms set forth. Although the invention has been described in detail with reference to certain embodiments, variations and modifications exist within the scope and spirit of the invention as described and defined in the following claims. The above disclosure and examples generally describe the present invention and are provided for purposes of illustration and is not intended to limit the scope of the invention.

The invention described herein may be practiced in the absence of any element or elements, limitation or limitations which is not specifically disclosed herein. Thus, for example, in each instance herein, any of the terms “comprising,” “consisting essentially of,” and “consisting of” may be replaced with either of the other two terms. The terms and expressions are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the claims.

1. A method for treating cutaneous radiation injury (CRI), said method comprising:
administering to a target area of skin of a patient in need thereof at least one time per day prior to, during, or after radiation treatment, 0.5-10 ml of a composition comprising an effective amount of one or more functional inhibitor of acid sphingomyelinase (FIASMA).

2. The method of claim 1 wherein treating CRI includes preventing, reducing the incidence or recurrence of, inhibiting, treating, ameliorating, or reducing the severity of, or reversing CRI.

3. The method of claim 1 wherein the CRI results from a medical radiation treatment for cancer.

4. The method of claim 1 wherein the CRI is radiation dermatitis.

5. The method of claim 1 wherein the CRI is oral mucositis.

6. The method of claim 1 wherein the CRI is radiation proctopathy.

7. The method of claim 1 wherein the composition is a topical cream, gel, or ointment.

8. The method of claim 1 wherein the composition is a solid oral dosage form.

9. The method of claim 8, wherein the solid oral dosage form is a mucoadhesive tablet.

10. The method of claim 1 wherein the composition is an oral solution.

11. The method of claim 10 wherein the oral solution is a rinse comprising 1-100 mg/ml of an SSRI.

12. The method of claim 1 wherein the FIASMA is selected from the group of a selective serotonin reuptake inhibitor (SSRI), tricyclic antidepressant (TCA), antihistamine, antiarrhythmic, adrenergic receptor blocker (ARB), and a beta blocker.

13. A pharmaceutical composition for treating cutaneous radiation injury (CRI), said composition comprising an effective amount of at least one functional inhibitor of acid sphingomyelinase (FIASMA).

14. The pharmaceutical composition of claim 13, wherein treating CRI includes preventing, reducing the incidence or recurrence of, inhibiting, treating, ameliorating, or reducing the severity of, or reversing CRI.

15. The pharmaceutical composition of claim 13, wherein the CRI is radiation dermatitis.

16. The pharmaceutical composition of claim 13, wherein the CRI is oral mucositis.

17. The pharmaceutical composition of claim 13, wherein the CRI is radiation proctopathy.

18. The pharmaceutical composition of claim 13 wherein the FIASMA is selected from the group of a selective serotonin reuptake inhibitor (SSRI), tricyclic antidepressant (TCA), tricyclic antihistamine, antiarrhythmic, adrenergic receptor blocker (ARB), and a beta blocker.

19. The composition of claim 13 wherein the composition is an oral dosage form comprising 1-100 mg/ml of an SSRI.

20. The composition of claim 19 wherein the oral dosage form is a mucoadhesive tablet.

21. A solid dosage form in the form of a lozenge or troche comprising an effective amount of loratadine for delivery to a patient undergoing radiation treatment of head and neck cancer.

22. The solid dosage form of claim 21 wherein the lozenge or troche comprises a handle affixed thereto to form a lollipop.

23. A method for preventing, treating or reducing the incidence of oral mucosits or sever oral mucositis (SOM) in a patient undergoing radiation treatment of head and neck cancer, said method comprising the steps of:
providing a lozenge or troche comprising loratadine of claim 25; and
administering the lozenge or troche to the patient as needed.
